(19) United States
(12) Patent Application Publication
(10) Pub. No.: US 2023/0149409 A1
(43) Pub. Date: May 18, 2023

(54) TREATMENT OF HUMAN PAPILLOMAVIRUS-ASSOCIATED CANCERS BY PD-L1 INHIBITORS

(71) Applicant: Incyte Corporation, Wilmington, DE (US)

(72) Inventors: Ryan Geschwindt, Telford, PA (US); Stephen Douglas Rubin, Southampton, NJ (US)

(21) Appl. No.: 17/951,696

(22) Filed: Sep. 23, 2022

Related U.S. Application Data
(60) Provisional application No. 63/248,025, filed on Sep. 24, 2021.

(51) Int. Cl. A61K 31/519; A61K 31/437; A61P 35/00; A61K 45/06

(52) U.S. Cl. CPC A61K 31/519; A61K 31/437; A61K 45/06; A61P 35/00

(57) ABSTRACT

The present disclosure relates to the treatment of HPV-associated cancers by small molecule PD-L1 inhibitors.

### CROSS-REFERENCE TO RELATED APPLICATIONS

This application claims priority to U.S. Provisional Appl. No. 63/248,025, filed Sep. 24, 2021. The content of the prior application is incorporated by reference herein in its entirety.

### FIELD OF THE INVENTION

The present disclosure relates to the treatment of human papillomavirus (HPV)-associated cancers by small molecule PD-L1 inhibitors.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4): 195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-y treatment (Iwai et al, PNAS2002, 99(19):12293-7; Blank et al, Cancer Res 2004, 64(3): 1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-y production, as well as cell proliferation induced upon T cell receptor activation (Carter et al, Eur J Immunol 2002, 32(3):634-43; Freeman et al, J Exp Med 2000, 192(7): 1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and AP1 pathways, and for cytokine production such as IL-2, IFN-y and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al, Science 2001, 291:319-322). Using an LCMV model of chronic infection, it has been shown that PD-⅟PD-L1 interaction inhibits activation, expansion and acquisition of effector functions of virus-specific CD8 T cells (Barber et al, Nature 2006, 439, 682-7). Together, these data support the development of a therapeutic approach to block the PD-1-mediated inhibitory signaling cascade in order to augment or “rescue” T cell response.

Malignancies caused by HPV contribute to a large number of deaths across several tumor types including cervical cancer, head and neck squamous cell carcinomas (HNSCC), anal cancer, vulvar cancer, vaginal cancer, and penile cancer (S. Otter, et al., “The human papillomavirus as a common pathogen in oropharyngeal, anal and cervical cancers,” Clin Oncol 2019;31:81-90). Hence, there is a need for treatments for HPV-positive tumors. The present disclosure addresses this need and others.

### SUMMARY

The present disclosure provides, inter alia, methods of treating a human papillomavirus (HPV)-positive cancer in a human subject in need thereof, comprising administering to the human subject a small molecule PD-L1 inhibitor.

The present disclosure also provides methods of treating cancer in a human subject in need thereof, comprising administering to the human subject a small molecule PD-L1 inhibitor, wherein the cancer has been previously determined to be HPV-positive.

The present disclosure further provides methods of treating an HPV-positive cancer in a human subject in need thereof, comprising:
identifying a sample obtained from the human subject as positive for HPV; and
administering to the human subject a small molecule PD-L1 inhibitor.

In some embodiments of the foregoing methods, the PD-L1 inhibitor has a molecular weight of less than 1000 daltons.

In some embodiments of the foregoing methods, the PD-L1 inhibitor is a compound of Formula (I):

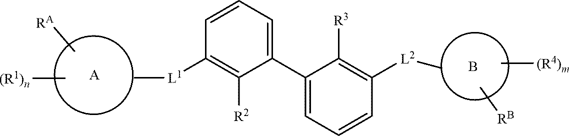

or a pharmaceutically acceptable salt thereof, wherein constituent variables are defined herein.

The present disclosure also provides a PD-L1 inhibitor for use in treatment of an HPV-positive cancer in a human subject.

The present disclosure further provides a PD-L1 inhibitor for use in treatment of an HPV-positive cancer in a human subject.

The present disclosure further provides a PD-L1 inhibitor for use in treatment of an HPV-positive cancer in a human subject, wherein a sample from the human subject has been previously identified as positive for HPV.

The present disclosure also provides use of a PD-L1 inhibitor for preparation of a medicament for use in treatment of a cancer in a human subject, wherein the cancer has been previously been determined to be HPV-positive.

The present disclosure further provides use of a PD-L1 inhibitor for preparation of a medicament for use in treatment of a cancer in a human subject, wherein the cancer has been previously been determined to be HPV-positive.

The present disclosure further provides use of a PD-L1 inhibitor for preparation of a medicament for use in treatment of an HPV-positive cancer in a human subject, wherein a sample from the human subject has been previously identified as positive for HPV.

All publications, patent applications, patents, and other references mentioned herein are incorporated by reference in their entirety. Unless otherwise defined, all technical and scientific terms used herein have the same meaning as commonly understood by one of ordinary skill in the art to which this invention belongs. In case of conflict, the present specification, including definitions, will control. Suitable methods and materials are described below, although methods and materials similar or equivalent to those described herein can also be used in the practice or testing of the present invention. The materials, methods, and examples are illustrative only and not intended to be limiting.

Other features and advantages of the invention will be apparent from the following detailed description, and from the claims.

### DETAILED DESCRIPTION

The present disclosure provides, inter alia, methods of treating a human papillomavirus (HPV)-positive cancer in a human subject in need thereof, comprising administering to the human subject a small molecule PD-L1 inhibitor.

The present disclosure further provides a method of treating cancer in a human subject in need thereof, comprising administering to the human subject a small molecule PD-L1 inhibitor, wherein the cancer has been previously determined to be HPV-positive.

In some embodiments, the HPV-positive cancer is positive for at least one of HPV type 16, 18, 31, 33, 35, 39, 45, 51, 52, 56, 58, 59, 66, or 68. In some embodiments, the HPV-positive cancer is positive for HPV type 16 and/or 18. In some embodiments, the HPV-positive cancer is positive for HPV type 16. In some embodiments, the HPV-positive cancer is positive for HPV type 18. In some embodiments, the HPV-positive cancer is positive for HPV type 31. In some embodiments, the HPV-positive cancer is positive for HPV type 33. In some embodiments, the HPV-positive cancer is positive for HPV type 35. In some embodiments, the HPV-positive cancer is positive for HPV type 39. In some embodiments, the HPV-positive cancer is positive for HPV type 45. In some embodiments, the HPV-positive cancer is positive for HPV type 51. In some embodiments, the HPV-positive cancer is positive for HPV type 52. In some embodiments, the HPV-positive cancer is positive for HPV type 56. In some embodiments, the HPV-positive cancer is positive for HPV type 58. In some embodiments, the HPV-positive cancer is positive for HPV type 59. In some embodiments, the HPV-positive cancer is positive for HPV type 66. In some embodiments, the HPV-positive cancer is positive for HPV type 68. In some embodiments, the HPV-positive cancer is positive for p16.

The present disclosure is further directed to a method of treating an HPV-positive cancer in a human subject in need thereof, comprising:
identifying a sample obtained from the human subject as positive for HPV; and
administering to the human subject a small molecule PD-L1 inhibitor.

In some embodiments, the method comprises identifying the sample as positive for at least one of HPV type 16, 18, 31, 33, 35, 39, 45, 51, 52, 56, 58, 59, 66, or 68. In some embodiments, the method comprises identifying the sample as positive for HPV type 16 and/or 18. In some embodiments, the method comprises identifying the sample as positive for HPV type 16. In some embodiments, the method comprises identifying the sample as positive for HPV type 18. In some embodiments, the method comprises identifying the sample as positive for HPV type 31. In some embodiments, the method comprises identifying the sample as positive for HPV type 33. In some embodiments, the method comprises identifying the sample as positive for HPV type 35. In some embodiments, the method comprises identifying the sample as positive for HPV type 39. In some embodiments, the method comprises identifying the sample as positive for HPV type 45. In some embodiments, the method comprises identifying the sample as positive for HPV type 51. In some embodiments, the method comprises identifying the sample as positive for HPV type 52. In some embodiments, the method comprises identifying the sample as positive for HPV type 56. In some embodiments, the method comprises identifying the sample as positive for HPV type 58. In some embodiments, the method comprises identifying the sample as positive for HPV type 59. In some embodiments, the method comprises identifying the sample as positive for HPV type 66. In some embodiments, the method comprises identifying the sample as positive for HPV type 68.

In some embodiments, the method comprises detecting an HPV nucleic acid in the sample. In some embodiments, the method comprises detecting an HPV protein in the sample. In some embodiments, the method comprises identifying the sample as positive for HPV by p16 immunohistochemistry.

In some embodiments of the foregoing methods, the HPV-positive cancer is a solid tumor.

In some embodiments, the solid tumor is selected from the group consisting of anal cancer, cervical cancer, vaginal cancer, a head and neck cancer, vulvar cancer, rectal cancer, penile cancer, rectovaginal cancer, nasopharyngeal cancer, and acinic cell parotid gland carcinoma. In some embodiments, the solid tumor is selected from the group consisting of anal cancer, cervical cancer, vaginal cancer, a head and neck cancer, vulvar cancer, rectal cancer, and penile cancer. In some embodiments, the solid tumor is anal cancer. In some embodiments, the solid tumor is cervical cancer. In some embodiments, the solid tumor is vaginal cancer. In some embodiments, the solid tumor is a head and neck cancer. In some embodiments, the head and neck cancer is oral cavity cancer. In some embodiments, the head and neck cancer is oropharyngeal cancer. In some embodiments, the solid tumor is vulvar cancer. In some embodiments, the solid tumor is rectal cancer. In some embodiments, the solid tumor is penile cancer. In some embodiments, the solid tumor is rectovaginal cancer. In some embodiments, the solid tumor is nasopharyngeal cancer. In some embodiments, the solid tumor is acinic cell parotid gland carcinoma

In some embodiments of the foregoing methods, the PD-L1 inhibitor has a molecular weight of less than 1000 daltons.

In some embodiments of the foregoing methods, the PD-L1 inhibitor is one of the compounds described in the embodiments below, or a pharmaceutically acceptable salt thereof.

In some embodiments, the PD-L1 inhibitor is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
N-(2-chloro-3′-(8-chloro-6-((2-hydroxyethylamino)methyl)-[1,2,4]triazolo[1,5-a]pyridin-2-yl)-2′-methylbiphenyl-3-yl)-5-((2-hydroxyethylamino)methyl)picolinamide;
(S)-1-((7-cyano-2-(3′-(3-(((S)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
(R)-1-((7-cyano-2-(3′-(3-(((S)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
(S)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid
N,N′-(2-chloro-2′-methylbiphenyl-3,3′-diyl)bis(5-((2-hydroxyethylamino) methyl)picolinamide);
(R)-1-((6-(2′-chloro-3′-(5-((3-hydroxypyrrolidin-1-yl)methyl)picolinamido)-2-methylbiphenyl-3-ylcarbamoyl)pyridin-3-yl)methyl)piperidine-4-carboxylic acid
(S)-1-((6-((2′-chloro-2-methyl-3′-(5-(pyrrolidin-1-ylmethyl)picolinamido)-[1,1′-biphenyl]-3-yl)carbamoyl)-4-methylpyridin-3-yl)methyl)piperidine-2-carboxylic acid;
trans 4-(2-(2-(2-chloro-3′-(3-(((R)-3-hydroxypyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)ethyl)cyclohexanecarboxylic acid;
cis-4-((2-(2-chloro-3′-(3-(((R)-3-hydroxy-3-methylpyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)methyl)cyclohexanecarboxylic acid;
(R)-4-(2-(2-chloro-3′-(7-((3-hydroxypyrrolidm-l-yl)methyl)pyrido[3,2-d]pyrimidin-4-ylamino)-2′-methylbiphenyl-3-ylcarbamoyl)-1-methyl-6,7-dihydro-1H-imidazo[4,5-c]pyridin-5(4H)-yl)-1-methylcyclohexanecarboxylic acid;
(R)-1-((8-((2-chloro-3′-(5-(N-ethyl-N-methylglycyl)-5,6-dihydro-4H-pyrrolo[3,4-d]thiazol-2-yl)-2′-methyl-[1,1′-biphenyl]-3-yl)amino)-1,7-naphthyridin-3-yl)methyl)pyrrolidine-3-carboxylic acid;
(R)-2-(dimethylamino)-1-(2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazol-5(6H)-yl)ethanone;
trans-4-((2-(2′-chloro-3′-(1,5-dimethyl-4,5,6,7-tetrahydro-1H-imidazo[4,5-c]pyridine-2-carboxamido)-2-methylbiphenyl-3-ylcarbamoyl)-1-methyl-6,7-dihydro-1H-imidazo[4,5-c]pyridin-5(4H)-yl)methyl)cyclohexane-1-carboxylic acid;
trans-4-(2-(2-((2′-chloro-3′-(1,5-dimethyl-4,5,6,7-tetrahydro-1H-imidazo[4,5-c]pyridine-2-carboxamido)-2-methyl-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
cis-4-((2-((2′-chloro-3′-(1,5-dimethyl-4,5,6,7-tetrahydro-1H-imidazo[4,5-c]pyridine-2-carboxamido)-2-methyl-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid;
cis-4-((2-((2-chloro-3′-(1,5-dimethyl-4,5,6,7-tetrahydro-1H-imidazo[4,5-c]pyridine-2-carboxamido)-2′-methyl-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid;
trans-4-(2-(2-((2′-chloro-2-cyano-3′-(1,5-dimethyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)cyclohexane-1-carboxylic acid;
trans-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6, 7-dihydro-1H-imidazo[4,5-c]pyridin-5(4H)-yl)methyl)cyclohexane-1-carboxylic acid;
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid;
4-(2-(2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid;
4-(2-(2-(2-chloro-3′-(5-(2-(isopropyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbiphenyl-3-yl)benzo [d] oxazol-5-yl)methyl)-3-methylpyrrolidine-3-carboxylic acid;
(R)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(S)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid);
4-((2-((3′-(5-(2-(4-carboxybicyclo[2.2.1]heptan-1-yl)ethyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)methyl)bicyclo[2.2.1]heptane-1-carboxylic acid;
4,4′-(((((2-chloro-2′-methyl-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid);
4,4′-(((((2-chloro-2′-cyano-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)blcyclo[2.2.1]heptane-1-carboxylic acid;
or a pharmaceutically acceptable salt thereof.

In some embodiments, the PD-L1 inhibitor is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

### PD-L1 Inhibitors

The PD-L1 inhibitors useful in the methods described herein can cause internalization of cell surface PD-L1. Reducing cell surface expression of PD-L1 results in reduced PD-L1 available for ligand engagement with PD-1 on an opposing cell and thereby reduces the inhibitory signaling that results from the PD-1-PD-L1 interaction. By reducing PD-1 inhibitory signaling, the PD-L1 inhibitors increase an immune response and can be used to treat an HPV-positive cancer. In some embodiments, the HPV-positive cancer is a solid tumor.

As used herein “internalization” refers to the transport of PD-L1 proteins from the surface of a cell to the interior of the cell. As used herein, a PD-L1 inhibitor induces PD-L1 internalization if it causes PD-L1 internalization in the CHO/PD-L1 internalization assay described in Example 3A of US 2018-0177870-A1 or causes PD-L1 internalization in primary cells from cancer patients as described in Example 12A of US 2018-0177870-A1. In some embodiments, the compound causes at least 50%, (e.g., at least 60%, at least 70%, at least 75%, at least 80%, at least 85%, at least 90%, at least 95%, at least 98%, or at least 99%) of cell surface PD-L1 to be internalized. In some embodiments, a compound induces PD-L1 internalization if it causes PD-L1 internalization in the MDA-MB231/PD-L1 internalization assay described in Example 3A of US 2018-0177870-A1, which is incorporated herein by reference in its entirety. In some embodiments, a compound induces PD-L1 internalization if it causes PD-L1 internalization in primary cells from cancer patients described in the example herein.

Internalization can optionally be measured in the whole blood indirect internalization assay described in Example 3A of US 2018-0177870-A1, which is incorporated herein by reference in its entirety.

In some embodiments, the PD-L1 inhibitor binds to cell surface PD-L1 and induces PD-L1 internalization. In some embodiments, the cell is an immune cell (e.g., a monocyte or macrophage) or a tumor cell.

In some embodiments of any of the methods described herein, the PD-L1 inhibitor induces PD-L1 dimerization, and the dimerization occurs prior to PD-L1 internalization.

As used herein, a PD-L1 inhibitor induces PD-L1 dimerization if it yields a score in the range of 1.75 to 2.29 in the PD-L1 homogeneous time-resolved fluorescence dimerization assay described in Example 2A of US 2018-0177870-A1, which is incorporated herein by reference in its entirety. In some embodiments of any of the methods described herein, the PD-L1 inhibitor induces PD-L1 dimerization with a score in the range of 2.0 to 2.2 in the PD-L1 homogeneous time-resolved fluorescence dimerization assay described in Example 2A of US 2018-0177870-A1.

In some embodiments of any of the methods described herein, the PD-L1 inhibitor inhibits binding between PD-L1 and PD-1. In some embodiments, the PD-L1 inhibitor inhibits binding between PD-L1 and PD-1 with an IC₅₀ of less than 10 nM, less than 1 nM, or less than 0.5 nM.

In some embodiments, the PD-L1 inhibitor has a molecular weight of less than 1000 daltons. In some embodiments, the PD-L1 inhibitor has a molecular weight between 300 and 700 daltons.

In some embodiments, the PD-L1 inhibitor can be selected from any of the compounds of the embodiments and examples disclosed in U.S. Pat. Publication Nos. US 2018-0179201-A1, US 2018-0179197-A1, US 2018-0179179-A1, US 2018-0179202-A1, US 2018-0177784-A1, US 2018-0177870-A1, US 2019-0300524-A1, US 2019-0345170-A1, and US 2021-0094976-Al, each of which is incorporated herein by reference in its entirety, or a pharmaceutically acceptable salt thereof.

In some embodiments, the PD-L1 inhibitor is a salt, solid form, and crystalline form disclosed in U.S. Pat. Publication No. US-2021-0139511-A1, U.S. Pat. Publication No. US-2021-0040090-A1, U.S. Provisional No. 63/110,792, or U.S. Provisional No. 63/110,733, each of which is incorporated herein by reference in its entirety.

In some embodiments, the PD-L1 inhibitor is selected from the compounds in Table 1, or a pharmaceutically acceptable salt thereof.

TABLE 1

| | | |
|---|---|---|
| Compound No. | US Publication Appl. No. | Name and Structure |
| 1 | US 2018-0179197, Example #24 | (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid |
| | | 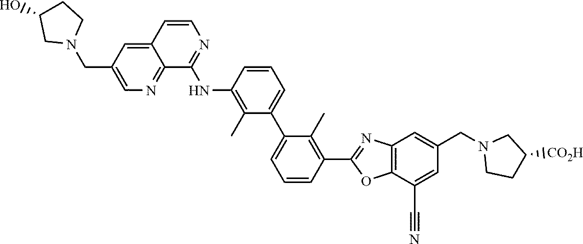 [SMILES: Cc1c(Nc2nccc3cc(CN4CC[C@@H](O)C4)cnc23)cccc1-c1cccc(-c2nc3cc(CN4CC[C@@H](C(=O)O)C4)cc(C#N)c3o2)c1C] |
| 2 | US 2018-0179201, Example #2 | N-(2-chloro-3′-(8-chloro-6-((2-hydroxyethylamino)methyl)-[1,2,4]triazolo[1,5-a]pyridin-2-yl)-2′-methylbiphenyl-3-yl)-5-((2-hydroxyethylamino)methyl)picolinamide |
| | | 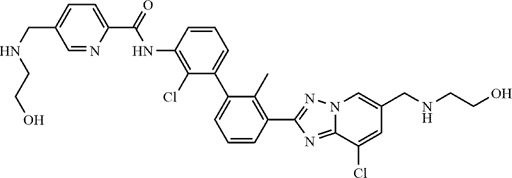 [SMILES: Cc1c(-c2nc3c(Cl)cc(CNCCO)cn3n2)cccc1-c1cccc(NC(=O)c2ccc(CNCCO)cn2)c1Cl] |
| 3 | US 2018-0179197, Example #25 | (S)-1-((7-cyano-2-(3′-(3-(((S)-3-hydroxypyrrolidin-1-yl)methyl)-1)-naphthyridin-8-ylamino)-2,2′-dimethylbiphenyl-3-yl)benzo[d]oxazol-5-yl)methyl)pyrrolidine-3-carboxylic acid |
| | | 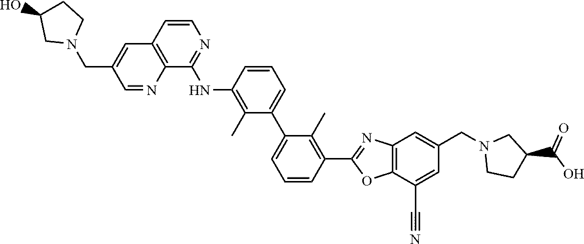 [SMILES: Cc1c(Nc2nccc3cc(CN4CC[C@H](O)C4)cnc23)cccc1-c1cccc(-c2nc3cc(CN4CC[C@H](C(=O)O)C4)cc(C#N)c3o2)c1C] |
| 4 | US 2018-0179197, Example #26 | (R)-1-((7-cyano-2-(3′-(3-(((S)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid |
| | | 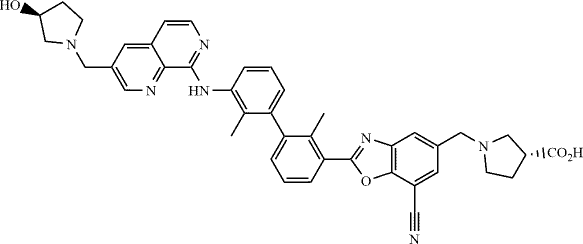 [SMILES: Cc1c(Nc2nccc3cc(CN4CC[C@H](O)C4)cnc23)cccc1-c1cccc(-c2nc3cc(CN4CC[C@@H](C(=O)O)C4)cc(C#N)c3o2)c1C] |
| 5 | US 2018-0179197, Example #28 | (S)-1-((7-cyano-2-(3′-(3-(((R)-3-hydroxypyrrolidin-1-yl)methyl)-1)-naphthyridin-8-ylamino)-2,2′-dimethylbiphenyl-3-yl)benzo[d]oxazol-5-yl)methyl)pyrrolidine-3-carboxylic acid |
| | | 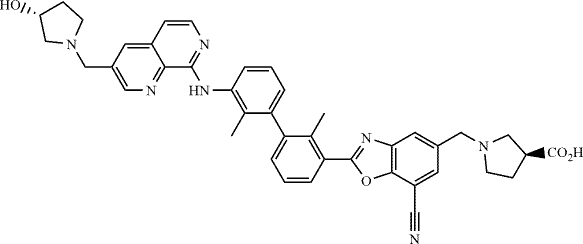 [SMILES: Cc1c(Nc2nccc3cc(CN4CC[C@@H](O)C4)cnc23)cccc1-c1cccc(-c2nc3cc(CN4CC[C@H](C(=O)O)C4)cc(C#N)c3o2)c1C] |
| 6 | US 2018-0179197, Example #236 | 1-((7-cyano-2-(3′-(5-(2-(dimethylamino)acetyl)-5,6-dihydro-4H-pyrrolo [3,4-d]thiazol-2-yl)-2,2′-dimethylbiphenyl-3-yl)benzo[d]oxazol-5-yl)methyl)piperidine-4-carboxylic acid |
| | | 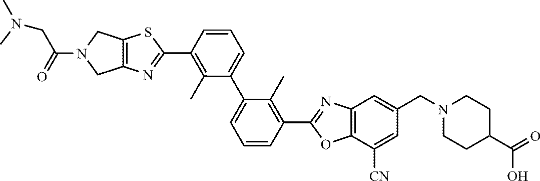 [SMILES: Cc1c(-c2nc3cc(CN4CCC(C(=O)O)CC4)cc(C#N)c3o2)cccc1-c1cccc(-c2nc3c(s2)CN(C(=O)CN(C)C)C3)c1C] |
| 7 | US 2018-0179179, Example #1 | N,N′-(2-chloro-2′-methylbiphenyl-3,3′-diyl)bis(5-((2-hydroxyethylamino) methyl)picolinamide) |
| | | 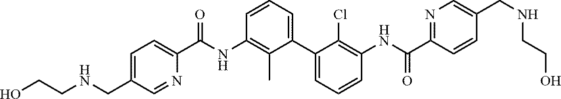 [SMILES: Cc1c(NC(=O)c2ccc(CNCCO)cn2)cccc1-c1cccc(NC(=O)c2ccc(CNCCO)cn2)c1Cl] |
| 8 | US 2018-0179179, Example #9 | (R)-1-((6-(2′-chloro-3′-(5-((3-hydroxypyrrolidin-1-yl)methyl)picolinamido)-2-methylbiphenyl-3 -ylcarbamoyl)pyridin-3 -yl)methyl)piperidine-4-carboxylic acid |
| | | 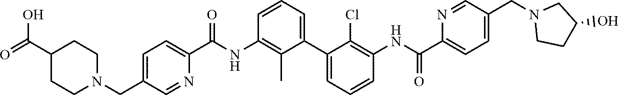 [SMILES: Cc1c(NC(=O)c2ccc(CN3CCC(C(=O)O)CC3)cn2)cccc1-c1cccc(NC(=O)c2ccc(CN3CC[C@@H](O)C3)cn2)c1Cl] |
| 9 | US 2018-0179179, Example #12 | (S)-1-((6-((2′-chloro-2-methyl-3′-(5-(pyrnolidin-1-ylmethyl)picolinamido)-[1,1′-biphenyl]-3-yl)carbamoyl)-4-methylpyridin-3-yl)methyl)piperidine-2-carboxylic acid |
| | | 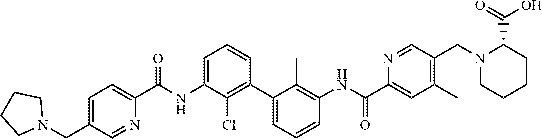 [SMILES: Cc1cc(C(=O)Nc2cccc(-c3cccc(NC(=O)c4ccc(CN5CCCC5)cn4)c3Cl)c2C)ncc1CN1CCCC[C@H]1C(=O)O] |
| 10 | US 2018-0179202, Example #52 | trans 4-(2-(2-(2-chloro-3′-(3-(((R)-3-hydroxypyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)ethyl)cyclohexanecarboxylic acid |
| | | 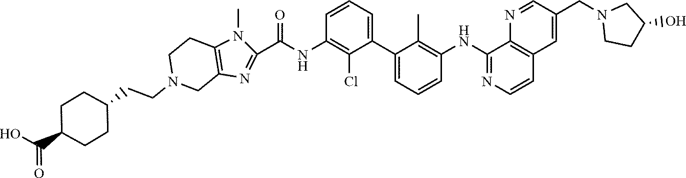 [SMILES: Cc1c(Nc2nccc3cc(CN4CC[C@@H](O)C4)cnc23)cccc1-c1cccc(NC(=O)c2nc3c(n2C)CCN(CC[C@H]2CC[C@H](C(=O)O)CC2)C3)c1Cl] |
| 11 | US 2018-0179202, Example #56 | cis-4-((2-(2-chloro-3′-(3-(((R)-3-hydroay-3-methylpyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)methyl)cyclohexanecarboxylic acid |
| | | 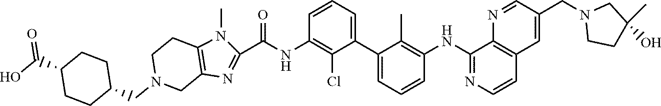 [SMILES: Cc1c(Nc2nccc3cc(CN4CC[C@@](C)(O)C4)cnc23)cccc1-c1cccc(NC(=O)c2nc3c(n2C)CCN(C[C@H]2CC[C@@H](C(=O)O)CC2)C3)c1Cl] |
| 12 | US 2018-0179202, Example #68 | (R)-4-(2-(2-chloro-3′-(7-((3-hydroxypyrrolidin-1-yl)methyl)pyrido[3,2-d]pyrimidin-4-ylamino)-2′-methylbiphenyl-3-ylcarbamoyl)-1-methyl-6,7-dihydro-1H-imidazof4,5-c]pyridin-5(4H)-yl)-1-methylcyclohexanecarboxylic acid |
| | | 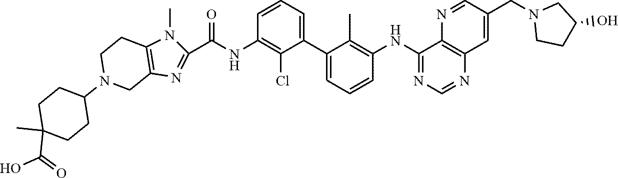 [SMILES: Cc1c(Nc2ncnc3cc(CN4CC[C@@H](O)C4)cnc23)cccc1-c1cccc(NC(=O)c2nc3c(n2C)CCN(C2CCC(C)(C(=O)O)CC2)C3)c1Cl] |
| 13 | US 2018-0179202, Example #90 | (R)-1-((8-((2-chloro-3′-(5-(N-ethyl-N-methylglycyl)-5,6-dihydro-4H-pyrrolo[3,4-d]thiazol-2-yl)-2′-methyl-[1,1′-biphenyl]-3-yl)amino)-1,7-naphthyridin-3-yl)methyl)pyrrolidine-3-carboxylic acid |
| | | 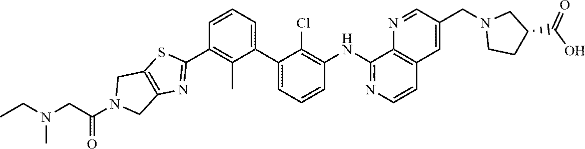 [SMILES: CCN(C)CC(=O)N1Cc2nc(-c3cccc(-c4cccc(Nc5nccc6cc(CN7CC[C@@H](C(=O)O)C7)cnc56)c4Cl)c3C)sc2C1] |
| 14 | US 2018-0177784, Example #35 | (R)-2-(dimethylamino)-1-(2-(3′-(5-(2-(3-hydroaypyrrolidin-1-yl)acetyl)-5,6-dihydro-4H-pyrrolo[3,4-d]thiazol-2-yl)-2,2′-dimethylbipheiryl-3-yl)-4H-pyrrolo [3,4-d]thiazol-5(6H)-yl)ethanone |
| | | 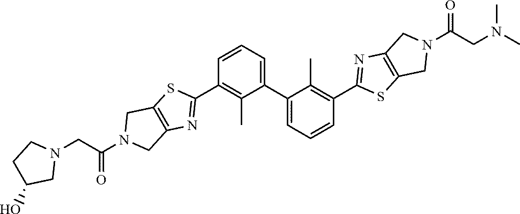 [SMILES: Cc1c(-c2nc3c(s2)CN(C(=O)CN(C)C)C3)cccc1-c1cccc(-c2nc3c(s2)CN(C(=O)CN2CC[C@@H](O)C2)C3)c1C] |
| 15 | US 2018-0177870, Example #37 | trans-4-((2-(2′-chloro-3′-(1,5-dimethyl-4,5,6,7-tetrahydro-1H-imidazo[4,5-c]pyfidine-2-caiboxamido)-2-methylbiphenyl-3-ylcarbamoyl)-1-methyl-6,7-dihydro-1H-imidazo[4,5-c]pyridin-5(4H)-yl)methyl)cyclohexane-1-carboxylic acid |
| | | 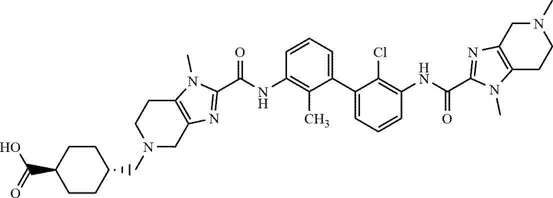 [SMILES: Cc1c(NC(=O)c2nc3c(n2C)CCN(C[C@H]2CC[C@H](C(=O)O)CC2)C3)cccc1-c1cccc(NC(=O)c2nc3c(n2C)CCN(C)C3)c1Cl] |
| 16 | US 2018-0177870, Example #100 | trans-4-(2-(2-((2′-chloro-3′-(1,5-dimethyl-4,5,6,7-tetrahydro-1H-imidazo[4,5-c]pyfidine-2-caiboxamido)-2-methyl-[1,1′-biphenyl]-3-yl)caibamoyl)-1-methyl-1,4,6,7-tetrahydro-5H-imidazo[4,5-c]pyridin-5-yl)ethyl)cyclohexane-1-carboxylic acid |
| | | 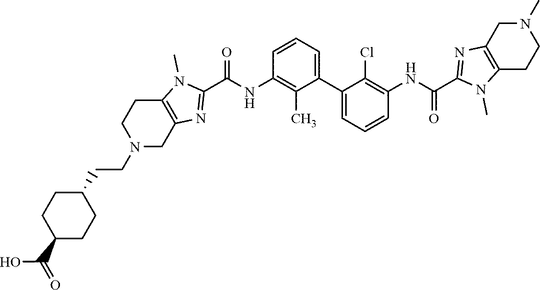 [SMILES: Cc1c(NC(=O)c2nc3c(n2C)CCN(CC[C@H]2CC[C@H](C(=O)O)CC2)C3)cccc1-c1cccc(NC(=O)c2nc3c(n2C)CCN(C)C3)c1Cl] |
| 17 | US 2018-0177870, Example #114 | cis-4-((2-((2′-chloro-3′-(1,5-dimethyl-4,5,6,7-tetrahydro-1H-imidazo[4,5-c]pyfidine-2-caiboxamido)-2-methyl-[1,1′-biphenyl]-3-yl)caibamoyl)-1-methyl-1,4,6,7-tetrahydro-5H-imidazo[4,5-c]pyridin-5-yl)methyl)cyclohexane-1-carboxylic acid |
| | | 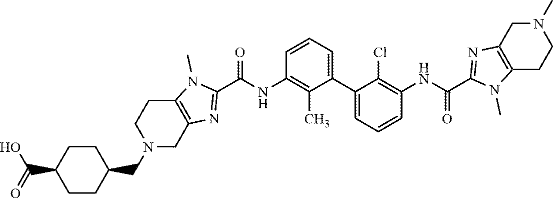 [SMILES: Cc1c(NC(=O)c2nc3c(n2C)CCN(C[C@H]2CC[C@@H](C(=O)O)CC2)C3)cccc1-c1cccc(NC(=O)c2nc3c(n2C)CCN(C)C3)c1Cl] |
| 18 | US 2018-0177870, Example #135 | cis-4-((2-((2-chloro-3′-(1,5-dimethyl-4,5,6,7-tetrahydro-1H-imidazo[4,5-c]pyfidine-2-caiboxamido)-2′-methyl-[1,1′-biphenyl]-3-yl)carbamoyl)-1-methyl-1,4,6,7-tetrahydro-5H-imidazo[4,5-c]pyridin-5-yl)methyl)cyclohexane-1-carboxylic acid |
| | | 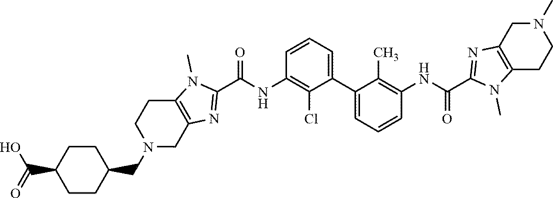 [SMILES: Cc1c(NC(=O)c2nc3c(n2C)CCN(C)C3)cccc1-c1cccc(NC(=O)c2nc3c(n2C)CCN(C[C@H]2CC[C@@H](C(=O)O)CC2)C3)c1Cl] |
| 19 | US 2018-0177870, Example #148 | trans-4-(2-(2-((2′-chloro-2-cyano-3′-(1,5-dimethyl-4,5,6,7-tetrahydro-1H-imidazo[4,5-c]pyridine-2-carboxamido)-[1,1′-biphenyl]-3yl)carbamoyl)-1-methyl-1,4,6,7-tetrahydro-5H-imidazo[4,5-c]pyridin-5-yl)ethyl)cyclohexane-1-carboxylic acid |
| | | 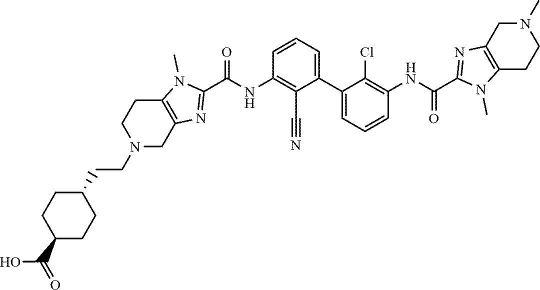 [SMILES: CN1CCc2c(nc(C(=O)Nc3cccc(-c4cccc(NC(=O)c5nc6c(n5C)CCN(CC[C@H]5CC[C@H](C(=O)O)CC5)C6)c4C#N)c3Cl)n2C)C1] |
| 20 | US 2018-0177870, Example #159 | trans-4-((2-(2-chloro-3′-(5-(2-(ethyl(methyl)amino)acetyl)-5,6-dihydro-4H-pyrrolo[3,4-d]thiazol-2-yl)-2′-methylbipheiryl-3-ylcarbamoyl)-1-methyl-6,7-dihydro-1H-imidazo [4,5-c]pyridin-5 (4H)-yl)methyl)cyclohexane-1-carboxylic acid |
| | | 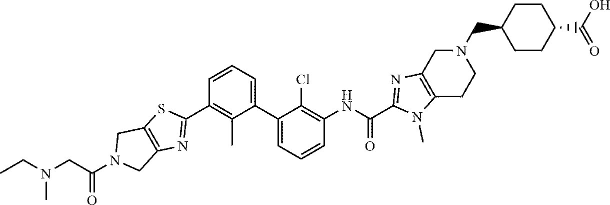 [SMILES: CCN(C)CC(=O)N1Cc2nc(-c3cccc(-c4cccc(NC(=O)c5nc6c(n5C)CCN(C[C@H]5CC[C@H](C(=O)O)CC5)C6)c4Cl)c3C)sc2C1] |
| 21 | US 2018-0177870, Example #160 | cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo [4,5-c]pyridin-5 (4H)-yl)methyl)cyclohexane-1-carboxylic acid |
| | | 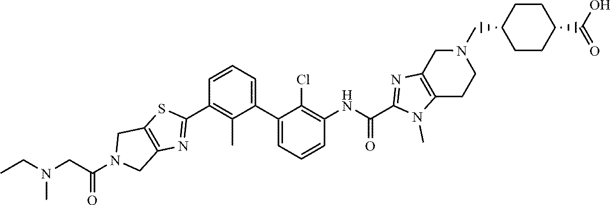 [SMILES: CCN(C)CC(=O)N1Cc2nc(-c3cccc(-c4cccc(NC(=O)c5nc6c(n5C)CCN(C[C@H]5CC[C@@H](C(=O)O)CC5)C6)c4Cl)c3C)sc2C1] |
| 22 | US 2018-0177870, Example #161 | 4-(2-(2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid |
| | | 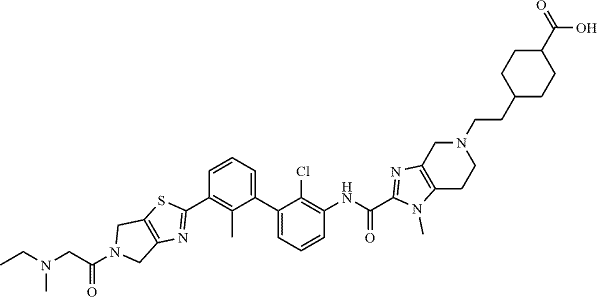 [SMILES: CCN(C)CC(=O)N1Cc2nc(-c3cccc(-c4cccc(NC(=O)c5nc6c(n5C)CCN(CCC5CCC(C(=O)O)CC5)C6)c4Cl)c3C)sc2C1] |
| 23 | US 2018-0177870, Example #162 | 4-(2-(2-(2-chloro-3′-(5-(2-(isopropyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid |
| | | 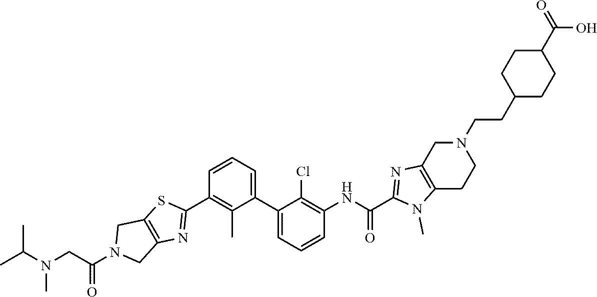 [SMILES: Cc1c(-c2nc3c(s2)CN(C(=O)CN(C)C(C)C)C3)cccc1-c1cccc(NC(=O)c2nc3c(n2C)CCN(CCC2CCC(C(=O)O)CC2)C3)c1Cl] |
| 24 | US 2019-0300524, Example #16 | (R)-1-((7-cyano-2-(3′-(2-(difluoromethyl)-7-((3-hydroxypyrrolidin-1-yl)methyl)pyirido[3,2-d]pyfimidin-4-ylamino)-2,2′-dimethylbiphenyl-3-yl(benzo[d]oxazol-5-yl)methyl)pipendine-4-carboxylic acid |
| | | 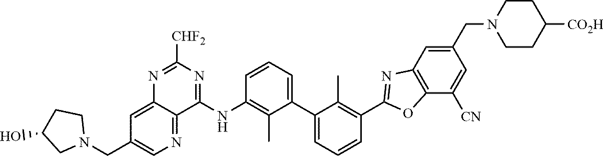 [SMILES: Cc1c(Nc2nc(C(F)F)nc3cc(CN4CC[C@@H](O)C4)cnc23)cccc1-c1cccc(-c2nc3cc(CN4CCC(C(=O)O)CC4)cc(C#N)c3o2)c1C] |
| 25 | US 2019-0300524, Example #17 | (R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbiphenyl-3-yl)benzo [d]oxazol-5-yl)methyl)pyrrolidine-3-carboxylic acid |
| | | 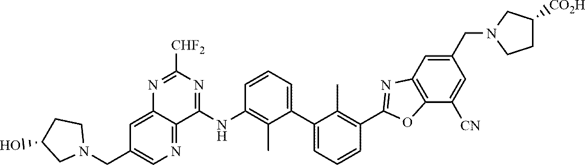 [SMILES: Cc1c(Nc2nc(C(F)F)nc3cc(CN4CC[C@@H](O)C4)cnc23)cccc1-c1cccc(-c2nc3cc(CN4CC[C@@H](C(=O)O)C4)cc(C#N)c3o2)c1C] |
| 26 | US 2019-0300524, Example #18 | (R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbipheiryl-3-yl)benzo[d]oxazol-5-yl)methyl)-3-methylpyrrolidine-3-carboxylic acid |
| | | 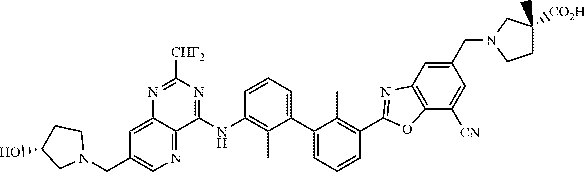 [SMILES: Cc1c(Nc2nc(C(F)F)nc3cc(CN4CC[C@@H](O)C4)cnc23)cccc1-c1cccc(-c2nc3cc(CN4CC[C@@](C)(C(=O)O)C4)cc(C#N)c3o2)c1C] |
| 27 | US 2019-0300524, Example #30 | (R)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbipheiryl-3-yl)benzo[d]oxazol-5-yl)methyl)piperidine-4-caiboxylic acid |
| | | 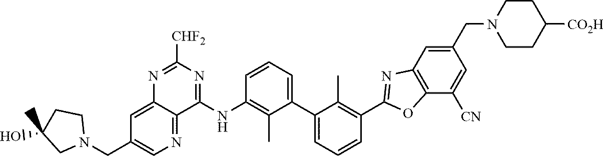 [SMILES: Cc1c(Nc2nc(C(F)F)nc3cc(CN4CC[C@@](C)(O)C4)cnc23)cccc1-c1cccc(-c2nc3cc(CN4CCC(C(=O)O)CC4)cc(C#N)c3o2)c1C] |
| 28 | US 2019-0300524, Example #31 | (S)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbipheiryl-3-yl)benzo[d]oxazol-5-yl)methyl)pipendine-4-carboxylic acid |
| | | 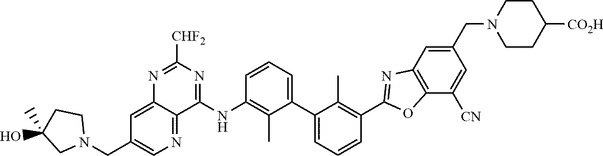 [SMILES: Cc1c(Nc2nc(C(F)F)nc3cc(CN4CC[C@](C)(O)C4)cnc23)cccc1-c1cccc(-c2nc3cc(CN4CCC(C(=O)O)CC4)cc(C#N)c3o2)c1C] |
| 29 | US 2019-0345170, Example #13 | (R)-4-(2-(2-((2,2′-dichloro-3′-(5-(2-hydroxypropyl)-1-methyl-4,5,6,7-tetrahydro-1H-imidazo[4,5-clpyfidine-2-carboxamido)-[1,1′-biphenyl]-3-yl)caibamoyl)-1-methyl-1,4,6,7-tetrahydro-5H-imidazo[4,5-c]pyridin-5-yl)ethyl)bicyclo[2.2.1]heptane-1-carboxylic acid |
| | | 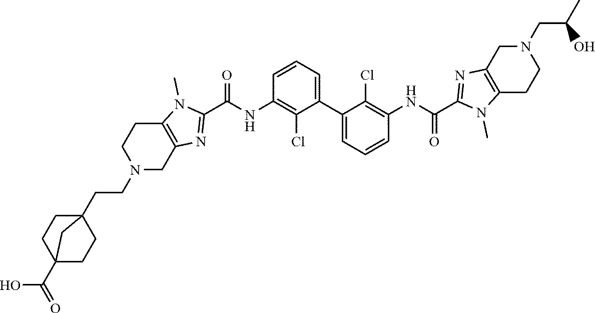 [SMILES: C[C@@H](O)CN1CCc2c(nc(C(=O)Nc3cccc(-c4cccc(NC(=O)c5nc6c(n5C)CCN(CCC57CCC(C(=O)O)(CC5)C7)C6)c4Cl)c3Cl)n2C)C1] |
| 30 | US 2019-0345170, Example #17 | 4,4′-(((((2,2′-dichloro-[1,1′-bipheiryl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) |
| | | 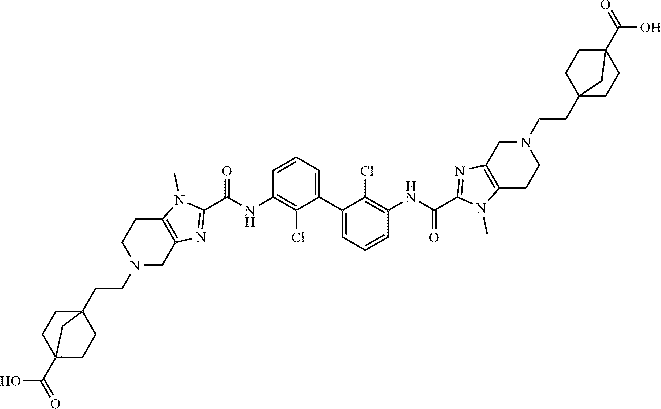 [SMILES: Cn1c(C(=O)Nc2cccc(-c3cccc(NC(=O)c4nc5c(n4C)CCN(CCC46CCC(C(=O)O)(CC4)C6)C5)c3Cl)c2Cl)nc2c1CCN(CCC13CCC(C(=O)O)(CC1)C3)C2] |
| 31 | US 2019-0345170, Example #18 | 4-((2-((3′-(5-(2-(4-carboxybicyclo[2.2.1]heptan-1-yl)ethyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)methyl)bicyclo[2.2.1]heptane-1-carboxylic acid |
| | | 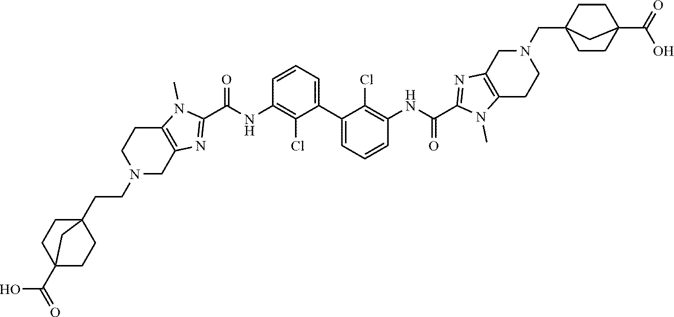 [SMILES: Cn1c(C(=O)Nc2cccc(-c3cccc(NC(=O)c4nc5c(n4C)CCN(CC46CCC(C(=O)O)(CC4)C6)C5)c3Cl)c2Cl)nc2c1CCN(CCC13CCC(C(=O)O)(CC1)C3)C2] |
| 32 | US 2019-0345170, Example #34 | 4,4′-(((((2-chloro-2′-methyl-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) |
| | | 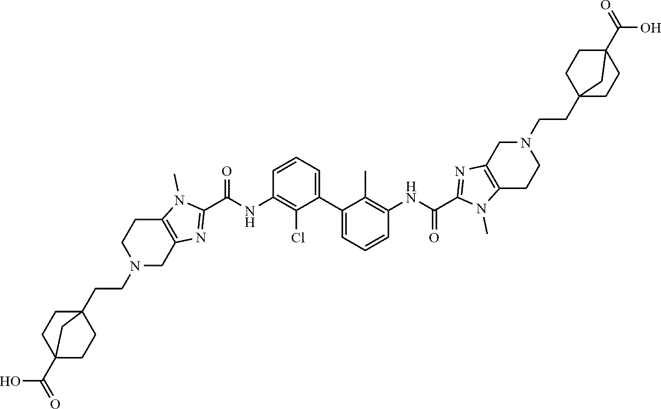 [SMILES: Cc1c(NC(=O)c2nc3c(n2C)CCN(CCC24CCC(C(=O)O)(CC2)C4)C3)cccc1-c1cccc(NC(=O)c2nc3c(n2C)CCN(CCC24CCC(C(=O)O)(CC2)C4)C3)c1Cl] |
| 33 | US 2019-0345170, Example #51 | 4,4′-(((((2-chloro-2′-cyano-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid) |
| | | 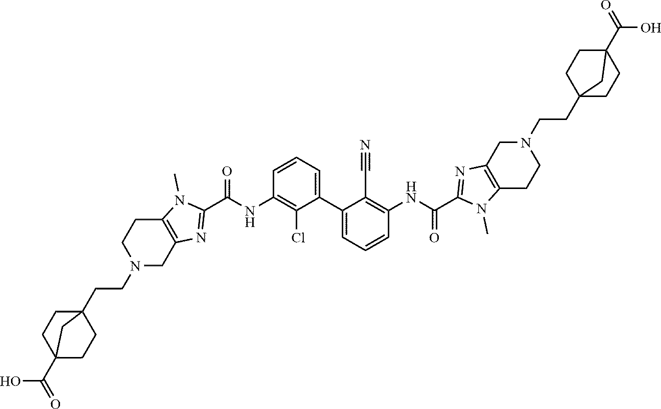 [SMILES: Cn1c(C(=O)Nc2cccc(-c3cccc(NC(=O)c4nc5c(n4C)CCN(CCC46CCC(C(=O)O)(CC4)C6)C5)c3C#N)c2Cl)nc2c1CCN(CCC13CCC(C(=O)O)(CC1)C3)C2] |
| 34 | US 2021-0094976, Example #1 | (R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid |
| | | 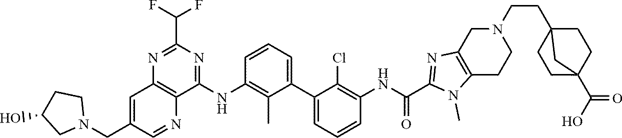 [SMILES: Cc1c(Nc2nc(C(F)F)nc3cc(CN4CC[C@@H](O)C4)cnc23)cccc1-c1cccc(NC(=O)c2nc3c(n2C)CCN(CCC24CCC(C(=O)O)(CC2)C4)C3)c1Cl] |

In some embodiments, the PD-L1 inhibitor is Compound 1 ((R)-1-((7-cyano-2-(3′-(3-(((R)-3-hydroxypyrrolidin-l-yl)methyl)-1,7-naphthyridin-8-ylamino)-2,2′-dimethylbiphenyl-3-yl)benzo[d]oxazol-5-yl)methyl)pyrrolidine-3-carboxylic acid), or a pharmaceutically acceptable salt thereof.

In some embodiments, the PD-L1 inhibitor is Compound 24 ((R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid), or a pharmaceutically acceptable salt thereof.

In some embodiments, the administering comprises administering 200 mg to 800 mg of Compound 24 twice daily (BID) to the human subject. In some embodiments, the administering comprises administering 400 mg to 800 mg of Compound 24 twice daily (BID) to the human subject. In some embodiments, the administering comprises administering 200 mg to 800 mg of Compound 24 once daily (QD) to the human subject. In some embodiments, the administering comprises administering 400 mg to 800 mg of Compound 24 once daily (QD) to the human subject. In some embodiments, the administering comprises administering 200 mg, 300 mg, 400 mg, 500 mg, 600 mg, 700 mg, or 800 mg of Compound 24 twice daily (BID) to the human subject. In some embodiments, the administering comprises administering 200 mg, 300 mg, 400 mg, 500 mg, 600 mg, 700 mg, or 800 mg of Compound 24 once daily (QD) to the human subject.

In some embodiments, the PD-L1 inhibitor is Compound 30 (4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid)), or a pharmaceutically acceptable salt thereof.

In some embodiments, the administering comprises administering 200 mg to 800 mg of Compound 30 twice daily (BID) to the human subject. In some embodiments, the administering comprises administering 400 mg to 800 mg of Compound 30 twice daily (BID) to the human subject. In some embodiments, the administering comprises administering 200 mg to 800 mg of Compound 30 once daily (QD) to the human subject. In some embodiments, the administering comprises administering 400 mg to 800 mg of Compound 30 once daily (QD) to the human subject. In some embodiments, the administering comprises administering 200 mg, 300 mg, 400 mg, 500 mg, 600 mg, 700 mg, or 800 mg of Compound 30 twice daily (BID) to the human subject. In some embodiments, the administering comprises administering 200 mg, 300 mg, 400 mg, 500 mg, 600 mg, 700 mg, or 800 mg of Compound 30 once daily (QD) to the human subject.

In some embodiments, the PD-L1 inhibitor is a compound of Formula (I):

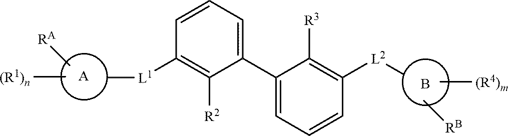

or a pharmaceutically acceptable salt thereof, wherein:
ring A is selected from 5-10-membered heteroaryl, having carbon ring members and 1, 2, 3, or 4 heteroatom ring members selected from N, O and S; and 4-10-membered heterocycloalkyl, having carbon ring members and 1, 2, 3, or 4 heteroatom ring members selected from N, O and S;
ring B is selected from 5-10-membered heteroaryl, having carbon ring members and 1, 2, 3, or 4 heteroatom ring members selected from N, O and S; and 4-10-membered heterocycloalkyl, having carbon ring members and 1, 2, 3, or 4 heteroatom ring members selected from N, O and S;
L¹ is a bond, —C(O)NH—, —NHC(O)-, and —NH—;
L² is a bond, —C(O)NH—, —NHC(O)—, and —NH—;
each R¹ is independently halo, CN, C₁₋₃ alkyl, C₁₋₃ alkoxy, C₁₋₃ haloalkyl, C₁₋₃ haloalkoxy, amino, C₁₋₃ alkylamino, or di(C₁₋₃ alkyl)amino;
R² is halo, CH₃, or CN;
R³ is halo, CH₃, or CN;
each R⁴ is independently halo, CN, C₁₋₃ alkyl, C₁₋₃ alkoxy, C₁₋₃ haloalkyl, C₁₋₃ haloalkoxy, amino, C₁₋₃ alkylamino, or di(C₁₋₃ alkyl)amino;
R^{A} and R^{B} are each independently selected from C₁₋₃ alkyl, C₄₋₇ cycloalkylene-(R^{c}), -(C₁₋₃ alkylene)-R^{c} and -C(=O)-(C₁₋₃ alkylene)-R^{c}, wherein said C₄₋₇-cycloalkylene is optionally substituted by one C₁₋₃ alkyl substituent;
each R^{c}is independently selected from phenyl, C₃₋₇ cycloalkyl, 5-6 membered heteroaryl, 4-10 membered heterocycloalkyl, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, NR^{c1}S(O)NR^{cl}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein the phenyl, C₃₋₇ cycloalkyl, 5-6 membered heteroaryl, and 4-10 membered heterocycloalkyl of R^{c} are each optionally substituted with 1, 2, or 3 independently selected R^{D} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl-, wherein said C₁₋₆ alkyl, C₂₋₆ alkenyl, C₂₋₆ alkynyl, C₁₋₆ haloalkyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl- are each optionally substituted with 1, 2, or 3 independently selected R^{D} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, or 3 independently selected R^{D} substituents;
each R^{b1} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl-, which are each optionally substituted with 1, 2, or 3 independently selected R^{D} substituents;
each R^{D} is independently selected from halo, C₁₋₆ alkyl, C₂₋₆ alkenyl, C₂₋₆ alkynyl, C₁₋₆haloalkyl, C₁₋₆haloalkoxy, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, (4-10 membered heterocycloalkyl)-C₁₋₄ alkyl-, CN, OH, NH₂, NO₂, OR^{a2}, SR^{a2}, NHOR^{a2}, C(O)R^{b2}, C(O)NR^{c2}R^{d2}, C(O)NR^{c2}(OR^{a2}), C(O)OR^{a2}, OC(O)R^{b2}, OC(O)NR^{C2}R^{d2}, NR^{c2}R^{d2}, NR^{c2}NR^{c2}R^{d2}, NR^{c2}C(O)R^{b2}, NR^{C2}C(O)OR^{a2}, NR^{c2}C(O)NRc²R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₁₋₆ alkyl, C₂₋₆ alkenyl, C₂₋₆ alkynyl, C₁₋₆ haloalkyl, C₁₋₆haloalkoxy, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl- of R^{D} are each further optionally substituted with 1, 2, or 3 independently selected R^{E} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, or 3 independently selected R^{E} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, or 3 independently selected R^{E} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, or 3 independently selected R^{E} substituents;
each R^{E} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO-C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
the subscript n is an integer of 0, 1, or 2; and
the subscript m is an integer of 0, 1, or 2.

In some embodiments, Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl.

In some embodiments, Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl.

In some embodiments, Ring A is selected from naphthyridin-8-yl, [1,2,4]triazolo[1,5-a]pyridin-2-yl, pyridin-2-yl, pyrido[3,2-d]pyrimidin-4-yl, and 5,6-dihydro-4H-pyrrolo[3,4-d]thiazol-2-yl.

In some embodiments, Ring A is benzo[d]oxazol-5-yl.

In some embodiments, Ring A is naphthyridin-8-yl.

In some embodiments, Ring A is pyrido[3,2-d]pyrimidin-4-yl.

In some embodiments, Ring A is [1,2,4]triazolo[1,5-a]pyridin-2-yl.

In some embodiments, Ring A is pyridin-2-yl.

In some embodiments, Ring A is 4,5,6,7-tetrahydro-1H-imidazo[4,5-c]pyridin-2-yl.

In some embodiments, Ring A is 5,6-dihydro-4H-pyrrolo[3,4-d]thiazol-2-yl

In some embodiments, Ring A is pyrazin-2-yl.

In some embodiments, Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl.

In some embodiments, Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl.

In some embodiments, Ring B is selected from benzo[d]oxazol-5-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl.

In some embodiments, Ring B is benzo[d]oxazol-5-yl.

In some embodiments, Ring B is naphthyridin-8-yl.

In some embodiments, Ring B is pyrido[3,2-d]pyrimidin-4-yl.

In some embodiments, Ring B is [1,2,4]triazolo[1,5-a]pyridin-2-yl.

In some embodiments, Ring B is pyridin-2-yl.

In some embodiments, Ring B is 4,5,6,7-tetrahydro-1H-imidazo[4,5-c]pyridin-2-yl.

In some embodiments, Ring B is 5,6-dihydro-4H-pyrrolo[3,4-d]thiazol-2-yl

In some embodiments, Ring B is pyrazin-2-yl.

In some embodiments, Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl; and Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl.

In some embodiments, Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl; and Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl.

In some embodiments, L¹ is a bond; and L² is a bond.

In some embodiments, L¹ is -NH-; and L² is a bond.

In some embodiments, L¹ is -C(O)NH-; and L² is -NHC(O)-.

In some embodiments, L¹ is -NH-; and L² is -NHC(O)-.

In some embodiments, L¹ is a bond; and L² is -NHC(O)-.

In some embodiments, R² and R³ are each independently Cl, CH₃, or CN.

In some embodiments, R² and R³ are each independently Cl or CH₃.

In some embodiments, R² and R³ are each independently CH₃.

In some embodiments, each R¹ and R⁴ are independently CHF₂, CN, Cl, OCH₃, or CH₃.

In some embodiments, each R¹ and R⁴ are independently CHF₂, CN, Cl, or CH₃.

In some embodiments, each R¹ is independently CHF₂, CN, Cl, OCH₃, or CH₃.

In some embodiments, each R¹ is independently CHF₂, CN, Cl, or CH₃.

In some embodiments, R¹ is CHF₂.

In some embodiments, R¹ is CH₃.

In some embodiments, each R⁴ is independently CHF₂, CN, Cl, OCH₃, or CH₃.

In some embodiments, each R⁴ is independently CHF₂, CN, Cl, or CH₃.

In some embodiments, R⁴ is CN.

In some embodiments, R⁴ is CH₃.

In some embodiments, n is 0 or 1; and m is 0 or 1.

In some embodiments, n is 0 or 1.

In some embodiments, m is 0 or 1.

In some embodiments, R^{A} and R^{B} are each independently selected from CH₃, -CH₂-R^{C}, -CH₂CH₂-R^{C}, -(C₃ alkylene)-R^{C}, -(cyclohex-1,4-diyl)-(R^{C}), and -C(=O)-CH₂-R^{C}, wherein said cyclohex-1,4-diyl is optionally substituted by one methyl substituent.

In some embodiments, R^{A} and R^{B} are each independently selected from CH₃, -CH₂-R^{C}, -CH₂CH₂-R^{C}, -CH₂-CH(R^{C})-CH₃, -(cyclohex-1,4-diyl)-(R^{C}), and -C(=O)-CH₂-R^{C}, wherein said cyclohex-1,4-diyl is optionally substituted by one methyl substituent.

In some embodiments, each R^{C} is independently selected from OR^{a1}, NR^{c1}R^{d1}, cyclohexyl, bicyclo[2.2.1]heptanyl, pyrrolidinyl, and piperidinyl, wherein the cyclohexyl, bicyclo[2.2.1]heptanyl, pyrrolidinyl, and piperidinyl of R^{C} are each optionally substituted with 1 or 2 independently selected R^{D} substituents.

In some embodiments, each R^{C} is pyrrolidinyl, which is optionally substituted with 1 R^{D} substituent.

In some embodiments, each R^{C} is independently piperidinyl or pyrrolidinyl, which is substituted with 1 R^{D} substituent.

In some embodiments, each R^{C} is pyrrolidinyl, which is substituted with 1 R^{D} substituent.

In some embodiments, each R^{C} is piperidinyl, which is substituted with 1 R^{D} substituent.

In some embodiments,
each R^{a1} is H; and
each R^{c1} and R^{d1} is independently selected from H, methyl, ethyl, and 2-oxo-pyrrolidinylmethyl, wherein said methyl and ethyl are each optionally substituted with 1 or 2 independently selected R^{D} substituents.

In some embodiments,
each R^{a1} is H; and
each R^{c1} and R^{d1} is independently selected from H, methyl, and ethyl, wherein said methyl and ethyl are each optionally substituted with 1 or 2 independently selected R^{D} substituents.

In some embodiments, each R^{D} is independently selected from OH, CO₂H, and CH₃.

In some embodiments, each R^{D} is independently selected from OH and CO₂H.

In some embodiments, the HPV-positive cancer is a solid tumor.

In some embodiments of any of the methods described herein, a second therapeutic agent (e.g., a chemotherapeutic, an immunomodulatory agent, or a kinase inhibitor) is administered in combination with the compound.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, -NR(CR′R″)ₙ- includes both -NR(CR′R″)ₙ- and -(CR′R″)ₙNR- and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms. As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula -NH₂.

As used herein, the term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. In some embodiments, the aryl group has 6 to 10 carbon atoms. In some embodiments, the aryl group is phenyl or naphthyl. In some embodiments, the aryl is phenyl.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, “Cₙ₋ₘ haloalkoxy” refers to a group of formula -O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group of the haloalkyl has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

As used herein, the term “thio” refers to a group of formula -SH.

As used herein, the term “Cₙ₋ₘ alkylamino” refers to a group of formula -NH(alkyl), wherein the alkyl group has n to m carbon atoms. In some embodiments, the alkyl group of the alkylamino has 1 to 6, 1 to 4, or 1 to 3 carbon atoms.

As used herein, the term “Cₙ₋ₘ alkoxy carbonyl” refers to a group of formula -C(O)O-alkyl, wherein the alkyl group has n to m carbon atoms. In some embodiments, the alkyl group of the alkoxycarbonyl has 1 to 6, 1 to 4, or 1 to 3 carbon atoms.

As used herein, the term “Cₙ₋ₘ alkylcarbonyl” refers to a group of formula -C(O)-alkyl, wherein the alkyl group has n to m carbon atoms. In some embodiments, the alkyl group of the alkylcarbonyl has 1 to 6, 1 to 4, or 1 to 3 carbon atoms.

As used herein, the term “Cₙ₋ₘ alkylcarbonylamino” refers to a group of formula -NHC(O)-alkyl, wherein the alkyl group has n to m carbon atoms. In some embodiments, the alkyl group of the alkylcarbonylamino has 1 to 6, 1 to 4, or 1 to 3 carbon atoms.

As used herein, the term “Cₙ₋ₘ alkoxycarbonylamino” refers to a group of formula -NHC(O)O(Cₙ₋ₘ alkyl), wherein the alkyl group has n to m carbon atoms. In some embodiments, the alkyl group of the alkoxycarbonylamino has 1 to 6, 1 to 4, or 1 to 3 carbon atoms.

As used herein, the term “Cₙ₋ₘ alkylsulfonylamino” refers to a group of formula -NHS(O)₂-alkyl, wherein the alkyl group has n to m carbon atoms. In some embodiments, the alkyl group of the alkylsulfonylamino has 1 to 6, 1 to 4, or 1 to 3 carbon atoms.

As used herein, the term “aminosulfonyl” refers to a group of formula -S(O)₂NH₂.

As used herein, the term “Cₙ₋ₘ alkylaminosulfonyl” refers to a group of formula -S(O)₂NH(alkyl), wherein the alkyl group has n to m carbon atoms. In some embodiments, the alkyl group of the alkylaminosulfonyl has 1 to 6, 1 to 4, or 1 to 3 carbon atoms.

As used herein, the term “di(Cₙ₋ₘ alkyl)aminosulfonyl” refers to a group of formula -S(O)₂N(alkyl)₂, wherein each alkyl group independently has n to m carbon atoms. In some embodiments, each alkyl group of the dialkylaminosulfonyl has, independently, 1 to 6, 1 to 4, or 1 to 3 carbon atoms.

As used herein, the term “aminosulfonylamino” refers to a group of formula - NHS(O)₂NH₂.

As used herein, the term “Cₙ₋ₘ alkylaminosulfonylamino” refers to a group of formula -NHS(O)₂NH(alkyl), wherein the alkyl group has n to m carbon atoms. In some embodiments, the alkyl group of the alkylaminosulfonylamino has 1 to 6, 1 to 4, or 1 to 3 carbon atoms.

As used herein, the term “di(Cₙ₋ₘ alkyl)aminosulfonylamino” refers to a group of formula -NHS(O)₂N(alkyl)₂, wherein each alkyl group independently has n to m carbon atoms. In some embodiments, each alkyl group of the dialkylaminosulfonylamino has, independently, 1 to 6, 1 to 4, or 1 to 3 carbon atoms.

As used herein, the term “aminocarbonylamino”, employed alone or in combination with other terms, refers to a group of formula -NHC(O)NH₂.

As used herein, the term “Cₙ₋ₘ alkylaminocarbonylamino” refers to a group of formula -NHC(O)NH(alkyl), wherein the alkyl group has n to m carbon atoms. In some embodiments, the alkyl group of the alkylaminocarbonylamino has 1 to 6, 1 to 4, or 1 to 3 carbon atoms.

As used herein, the term “di(Cₙ₋ₘ alkyl)aminocarbonylamino” refers to a group of formula -NHC(O)N(alkyl)₂, wherein each alkyl group independently has n to m carbon atoms. In some embodiments, each alkyl group of the dialkylaminocarbonylamino has, independently, 1 to 6, 1 to 4, or 1 to 3 carbon atoms.

As used herein, the term “Cₙ₋ₘ alkylcarbamyl” refers to a group of formula -C(O)-NH(alkyl), wherein the alkyl group has n to m carbon atoms. In some embodiments, the alkyl group of the alkylcarbamyl has 1 to 6, 1 to 4, or 1 to 3 carbon atoms.

As used herein, the term “Cₙ₋ₘ alkylthio” refers to a group of formula -S-alkyl, wherein the alkyl group has n to m carbon atoms. In some embodiments, the alkyl group of the alkylthio has 1 to 6, 1 to 4, or 1 to 3 atoms.

As used herein, the term “Cₙ₋ₘ alkylsulfinyl” refers to a group of formula -S(O)-alkyl, wherein the alkyl group has n to m carbon atoms. In some embodiments, the alkyl group of the alkylsulfinyl has 1 to 6, 1 to 4, or 1 to 3 carbon atoms.

As used herein, the term “Cₙ₋ₘ alkylsulfonyl” refers to a group of formula -S(O)₂-alkyl, wherein the alkyl group has n to m carbon atoms. In some embodiments, the alkyl group of the alkylsulfonyl has 1 to 6, 1 to 4, or 1 to 3 carbon atoms.

As used herein, the term “cyano-Cₙ₋ₘ alkyl” refers to a group of formula -(Cₙ₋ₘ alkylene)-CN, wherein the alkylene group has n to m carbon atoms. As used herein, the term “cyano-C₁₋₆ alkyl” refers to a group of formula -(C₁₋₆ alkylene)-CN. As used herein, the term “cyano-C₁₋₃ alkyl” refers to a group of formula -(C₁₋₃ alkylene)-CN.

As used herein, the term “HO-Cₙ₋ₘ alkyl” refers to a group of formula -(Cₙ₋ₘ alkylene)-OH, wherein the alkylene group has n to m carbon atoms. As used herein, the term “HO-C₁₋₃ alkyl” refers to a group of formula -(C₁₋₃ alkylene)-OH.

As used herein, the term “Cₙ₋ₘ alkoxy-Cₒ₋ₚ alkyl” refers to a group of formula -(Cₙ₋ₘ alkylene)-O(Cₒ₋ₚ alkyl), wherein the alkylene group has n to m carbon atoms and the alkyl group has o to p carbon atoms. As used herein, the term “C₁₋₆ alkoxy-C₁₋₆ alkyl” refers to a group of formula -(C₁₋₆ alkylene)-O(C₁₋₆ alkyl). As used herein, the term “C₁₋₃ alkoxy-C₁₋₃ alkyl” refers to a group of formula -(C₁₋₃ alkylene)-O(C₁₋₃ alkyl).

As used herein, the term “carboxy” refers to a group of formula -C(O)OH.

As used herein, the term “di(Cₙ₋ₘ-alkyl)amino” refers to a group of formula -N(alkyl)₂, wherein the two alkyl groups each has, independently, n to m carbon atoms. In some embodiments, each alkyl group of the dialkylamino independently has 1 to 6, 1 to 4, or 1 to 3 carbon atoms.

As used herein, the term “di(Cₙ₋ₘ-alkyl)carbamyl” refers to a group of formula -C(O)N(alkyl)₂, wherein the two alkyl groups each has, independently, n to m carbon atoms. In some embodiments, each alkyl group of the dialkylcarbamyl independently has 1 to 6, 1 to 4, or 1 to 3 carbon atoms.

As used herein, the term “Cₙ₋ₘ alkylcarbonyloxy” is a group of formula -OC(O)-alkyl, wherein the alkyl group has n to m carbon atoms. In some embodiments, the alkyl group of the alkylcarbonyloxy has 1 to 6, 1 to 4, or 1 to 3 carbon atoms.

As used herein, “aminocarbonyloxy” is a group of formula -OC(O)-NH₂.

As used herein, “Cₙ₋ₘ alkylaminocarbonyloxy” is a group of formula -OC(O)-NH-alkyl, wherein the alkyl group has n to m carbon atoms. In some embodiments, the alkyl group of the alkylaminocarbonyloxy has 1 to 6, 1 to 4, or 1 to 3 carbon atoms.

As used herein, “di(Cₙ₋ₘalkyl)aminocarbonyloxy” is a group of formula -OC(O)-N(alkyl)₂, wherein each alkyl group has, independently, n to m carbon atoms. In some embodiments, each alkyl group of the dialkylaminocarbonyloxy independently has 1 to 6, 1 to 4, or 1 to 3 carbon atoms.

As used herein “Cₙ₋ₘ alkoxycarbonylamino” refers to a group of formula -NHC(O)-O-alkyl, wherein the alkyl group has n to m carbon atoms.

As used herein, the term “carbamyl” to a group of formula -C(O)NH₂.

As used herein, the term “carbonyl”, employed alone or in combination with other terms, refers to a -C(O)- group.

As used herein, “cycloalkyl” refers to non-aromatic cyclic hydrocarbons including cyclized alkyl and alkenyl groups. Cycloalkyl groups can include mono- or polycyclic (e.g., having 2, 3 or 4 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄-₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, cubane, adamantane, bicyclo[1.1.1]pentyl, bicyclo[2.1.1]hexyl, bicyclo[2.2.1]heptanyl, bicyclo[3.1. 1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “cycloalkylene” refers to a divalent cycloalkyl moiety.

As used herein, “heteroaryl” refers to a monocyclic or polycyclic (e.g., having 2, 3, or 4 fused rings) aromatic heterocycle having at least one heteroatom ring member selected from N, O, or S. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, and S. In some embodiments, the heteroaryl group contains 5 to 10 or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, pyridyl, pyrimidinyl, pyrazinyl, pyridazinyl, pyrrolyl, pyrazolyl, azolyl, oxazolyl, isoxazolyl, thiazolyl, isothiazolyl, imidazolyl, furyl, thienyl, triazolyl (e.g., 1,2,3-triazolyl, 1,2,4-triazolyl, 1,3,4-triazolyl), tetrazolyl, thiadiazolyl (e.g., 1,2,3-thiadiazolyl, 1,2,4-thiadiazolyl, 1,3,4-thiadiazolyl), quinolinyl, isoquinolinyl, indolyl, benzothienyl, benzofuranyl, benzisoxazolyl, imidazo[1,2-b]thiazolyl, purinyl, triazinyl, thieno[3,2-b]pyridinyl, imidazo[1,2-α]pyridinyl, 1,5-naphthyridinyl, 1H-pyrazolo[4,3-b]pyridinyl, oxadiazolyl (e.g., 1,2,3-oxadiazolyl, 1,2,4-oxadiazolyl, 1,3,4-oxadiazolyl), 1,2-dihydro-1,2-azoborinyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, or S, and wherein the ring-forming carbon atoms and heteroatoms of the heterocycloalkyl group can be optionally substituted by one or more oxo or sulfido (e.g., C(O), S(O), C(S), or S(O)₂, etc.). Heterocycloalkyl groups include monocyclic and polycyclic (e.g., having 2 fused rings) systems. Included in heterocycloalkyl are monocyclic and polycyclic 4-10-, 4-7-, and 5-6-membered heterocycloalkyl groups. Heterocycloalkyl groups can also include spirocycles and bridged rings. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 4 to 10 ring-forming atoms, 4 to 7 ring-forming atoms, 4 to 6 ring-forming atoms or 5 to 6 ring-forming atoms. In some embodiments, the heterocycloalkyl group has 1 to 4 heteroatoms, 1 to 3 heteroatoms, 1 to 2 heteroatoms or 1 heteroatom.

In some embodiments, the heterocycloalkyl is a 4-10 membered monocyclic, bicyclic, or tricyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-10 membered bicyclic heterocycloalkyl having 1, 2, 3, or 4 ring-forming heteroatoms independently selected from N, O, and S, wherein 1, 2, 3, or 4 ring-forming carbon or heteroatoms can be optionally substituted by one or more oxo or sulfido. In some embodiments, the heterocycloalkyl is a 4-7 membered monocyclic heterocycloalkyl having 1 or 2 ring-forming heteroatoms independently selected from N, O, and S, and wherein 1, 2 or 3 ring-forming carbon or heteroatoms can be optionally substituted by one or more oxo or sulfido. In some embodiments, the heterocycloalkyl is a monocyclic 4-6 membered heterocycloalkyl having 1 or 2 heteroatoms independently selected from N, O, S, and B and having one or more oxidized ring members.

Example heterocycloalkyl groups include pyrrolidin-2-one, 1,3-isoxazolidin-2-one, pyranyl, tetrahydropyran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, 1,2,3,4-tetrahydroisoquinoline, azabicyclo[3.1.0]hexanyl, diazabicyclo[3.1.0]hexanyl, oxabicyclo[2.1.1]hexanyl, azabicyclo[2.2.1]heptanyl, diazabicyclo[2.2.1]heptanyl, azabicyclo[3.1.1]heptanyl, diazabicyclo[3.1. 1]heptanyl, azabicyclo[3.2.1]octanyl, diazabicyclo[3.2.1]octanyl, oxabicyclo[2.2.2]octanyl, azabicyclo[2.2.2]octanyl, azaadamantanyl, diazaadamantanyl, oxa-adamantanyl, azaspiro[3.3]heptanyl, diazaspiro[3.3]heptanyl, oxa-azaspiro[3.3]heptanyl, azaspiro[3.4]octanyl, diazaspiro[3.4]octanyl, oxa-azaspiro[3.4]octanyl, azaspiro[2.5]octanyl, diazaspiro[2.5]octanyl, azaspiro[4.4]nonanyl, diazaspiro[4.4]nonanyl, oxa-azaspiro[4.4]nonanyl, azaspiro[4.5]decanyl, diazaspiro[4.5]decanyl, diazaspiro[4.4]nonanyl, oxa-diazaspiro[4.4]nonanyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon ring members and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, the term “alkylene” refers to a divalent straight chain or branched alkyl linking group. Examples of “alkylene groups” include methylene, ethan-1,1-diyl, ethan-1,2-diyl, propan-1,3-dilyl, propan-1,2-diyl, propan-1,1-diyl and the like.

As used herein, the term “alkenylene” refers to a divalent straight chain or branched alkenyl linking group. Examples of “alkenylene groups” include ethen-1,1-diyl, ethen-1,2-diyl, propen-1,3-diyl, 2-buten-1,4-diyl, 3-penten-1,5-diyl, 3-hexen-1,6-diyl, 3-hexen-1,5-diyl, and the like.

As used herein, the term “alkynylene” refers to a divalent straight chain or branched alkynyl linking group. Examples of “alkynylene groups” include propyn-1,3-diyl, 2-butyn-1,4-diyl, 3-pentyn-1,5-diyl, 3-hexyn-1,6-diyl, 3-hexyn-1,5-diyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl or sulfonyl group.

As used herein, the term “independently selected from” means that each occurrence of a variable or substituent are independently selected at each occurrence from the applicable list.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the disclosure have the (R)-configuration. In other embodiments, the compounds have the (S)-configuration. In compounds with more than one chiral centers, each of the chiral centers in the compound may be independently (R) or (S), unless otherwise indicated.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone - enol pairs, amide - imidic acid pairs, lactam - lactim pairs, enamine - imine pairs, and annular forms where a proton can occupy two or more positions of a heterocyclic system, e.g., 1H- and 3H-imidazole, 1H-, 2H- and 4H- 1,2,4-triazole, 1H- and 2H- isoindole and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the disclosure can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art.

The term “compound,” as used herein is meant to include all stereoisomers, geometric isomers, tautomers and isotopes of the structures depicted. The term is also meant to refer to compounds of the disclosures, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the disclosure, or salts thereof, are substantially isolated. By “substantially isolated” is meant that the compound is at least partially or substantially separated from the environment in which it was formed or detected. Partial separation can include, e.g., a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, e.g., from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington’s Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Synthesis

Compounds of the disclosure, including salts thereof, can be prepared using known organic synthesis techniques and can be synthesized according to any of numerous possible synthetic routes.

The reactions for preparing compounds of the disclosure can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent’s freezing temperature to the solvent’s boiling temperature. A given reaction can be carried out in one solvent or a mixture of more than one solvent. Depending on the particular reaction step, suitable solvents for a particular reaction step can be selected by the skilled artisan.

Preparation of compounds of the disclosure can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March’s Advanced Organic Chemistry: Reactions, Mechanisms, and Structure, 6^{th} Ed. (Wiley, 2007); Petursson et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

In some embodiments, the PD-L1 inhibitor can be prepared by any of the synthetic processes described in U.S. Pat. Publication Nos. US 2018-0179201-A1, US 2018-0179197-A1, US 2018-0179179-A1, US 2018-0179202-A1, US 2018-0177784-A1, US 2018-0177870-A1, US 2019-0300524-A1, US 2019-0345170-A1, US 2021-0040090-A1, or US 2021-0094976-A1, each of which is incorporated herein by reference in its entirety, or a pharmaceutically acceptable salt thereof.

In some embodiments, the PD-L1 inhibitor can be prepared by any of the synthetic processes described in U.S. Pat. Publication No. US-2021-0139511-A1, U.S. Provisional No. 63/110,792, U.S. Provisional No. 63/110,733, or U.S. Provisional No. 63/110,779, each of which is incorporated herein by reference in its entirety.

### HPV-Positive Cancers

The PD-L1 inhibitors can be used alone, in combination with other agents or therapies or as an adjuvant or neoadjuvant for the treatment of an HPV-positive cancer. A compound described herein or of any of the formulas as described herein, or a compound as recited in any of the claims and described herein, or a salt or stereoisomer thereof, can be used to inhibit the growth of cancerous tumors. Alternatively, a compound of Formula (I) or of any of the formulas as described herein, or a compound as recited in any of the claims and described herein, or a salt or stereoisomer thereof, can be used in conjunction with other agents or standard cancer treatments, as described below. For the uses described herein, any of the compounds of the disclosure, including any of the embodiments thereof, may be used.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the disclosure are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Examples of HPV-positive cancers that are treatable using the methods of the present disclosure include, but are not limited to, solid tumors such as anal cancer, cervical cancer, vaginal cancer, head and neck cancer (e.g., oral cavity (mouth) cancer or oropharyngeal (throat) cancer), vulvar cancer, rectal cancer, penile cancer, rectovaginal cancer, nasopharyngeal cancer, and acinic cell parotid gland carcinoma.

The HPV status of a subject can be determined by analyzing a sample (e.g., a tissue sample) obtained from the subject for the presence of an HPV protein or nucleic acid or for the presence of a surrogate marker of HPV infection. For example, p16 immunohistochemistry (IHC) can be used to detect aberrant overexpression of the cell cycle protein p16 (CDKN2A) in a tissue sample, which overexpression results from an HPV-induced reduction of p53 and of functional Rb tumor-suppressor protein. As another example, HPV-type specific testing can be performed by using polymerase chain reaction (PCR) to detect an HPV nucleic acid. High risk HPV types include types 16, 18, 31, 33, 35, 39, 45, 51, 52, 56, 58, 59, 66, and 68 (see https://www.cancer.gov/about-cancer/causes-prevention/risk/infectious-agents/hpv-and-cancer). HPV types 16 and 18 in particular cause most HPV-related cancers, including about 70% of cervical cancers.

In some embodiments, the HPV-positive cancer treated is positive for at least one of HPV type 16, 18, 31, 33, 35, 39, 45, 51, 52, 56, 58, 59, 66, or 68. In some embodiments, the HPV-positive cancer is positive for HPV type 16. In some embodiments, the HPV-positive cancer is positive for HPV type 18. In some embodiments, the HPV-positive cancer is positive for HPV type 31. In some embodiments, the HPV-positive cancer is positive for HPV type 33. In some embodiments, the HPV-positive cancer is positive for HPV type 35. In some embodiments, the HPV-positive cancer is positive for HPV type 39. In some embodiments, the HPV-positive cancer is positive for HPV type 45. In some embodiments, the HPV-positive cancer is positive for HPV type 51. In some embodiments, the HPV-positive cancer is positive for HPV type 52. In some embodiments, the HPV-positive cancer is positive for HPV type 56. In some embodiments, the HPV-positive cancer is positive for HPV type 58. In some embodiments, the HPV-positive cancer is positive for HPV type 59. In some embodiments, the HPV-positive cancer is positive for HPV type 66. In some embodiments, the HPV-positive cancer is positive for HPV type 68.

In some embodiments, the HPV-positive cancer treated is positive for HPV type 16 and/or 18. In some embodiments, the HPV-positive cancer treated is positive for p16.

In some embodiments, an HPV-positive cancer is treated in a human subject by a method that entails: identifying a sample obtained from the human subject as positive for HPV (e.g., by a method described herein); and administering to the human subject a small molecule PD-L1 inhibitor. In some embodiments, the method entails identifying the sample as positive for at least one of HPV type 16, 18, 31, 33, 35, 39, 45, 51, 52, 56, 58, 59, 66, or 68. In some embodiments, the method comprises identifying the sample as positive for HPV type 16. In some embodiments, the method comprises identifying the sample as positive for HPV type 18. In some embodiments, the method comprises identifying the sample as positive for HPV type 31. In some embodiments, the method comprises identifying the sample as positive for HPV type 33. In some embodiments, the method comprises identifying the sample as positive for HPV type 35. In some embodiments, the method comprises identifying the sample as positive for HPV type 39. In some embodiments, the method comprises identifying the sample as positive for HPV type 45. In some embodiments, the method comprises identifying the sample as positive for HPV type 51. In some embodiments, the method comprises identifying the sample as positive for HPV type 52. In some embodiments, the method comprises identifying the sample as positive for HPV type 56. In some embodiments, the method comprises identifying the sample as positive for HPV type 58. In some embodiments, the method comprises identifying the sample as positive for HPV type 59. In some embodiments, the method comprises identifying the sample as positive for HPV type 66. In some embodiments, the method comprises identifying the sample as positive for HPV type 68. In some embodiments, the method entails identifying the sample as positive for HPV type 16 and/or 18. In some embodiments, the method entails identifying the sample as positive for HPV type 16. In some embodiments, the method entails identifying the sample as positive for HPV type 18.

In some embodiments, the method entails detecting an HPV nucleic acid in the sample. In some embodiments, the method entails detecting an HPV protein in the sample. In some embodiments, the method entails identifying the sample as positive for HPV by p16 immunohistochemistry. In some embodiments, the HPV-positive cancer is a solid tumor. In some embodiments, the solid tumor is selected from the group consisting of anal cancer, cervical cancer, vaginal cancer, a head and neck cancer, vulvar cancer, rectal cancer, penile cancer, rectovaginal cancer, nasopharyngeal cancer, and acinic cell parotid gland carcinoma. In some embodiments, the solid tumor is anal cancer. In some embodiments, the solid tumor is cervical cancer. In some embodiments, the solid tumor is vaginal cancer. In some embodiments, the solid tumor is a head and neck cancer. In some embodiments, the head and neck cancer is oral cavity cancer. In some embodiments, the head and neck cancer is oropharyngeal cancer. In some embodiments, the solid tumor is vulvar cancer. In some embodiments, the solid tumor is rectal cancer. In some embodiments, the solid tumor is penile cancer. In some embodiments, the solid tumor is rectovaginal cancer. In some embodiments, the solid tumor is nasopharyngeal cancer. In some embodiments, the solid tumor is acinic cell parotid gland carcinoma.

The cancer may be a carcinoma (e.g., carcinoma of the cervix) or a squamous cell carcinoma (e.g., squamous cell carcinoma of the vagina, vulva, penis, anus, rectum, or oropharynx). The cancer may be metastatic, especially metastatic cancers that express PD-L1.

### Combination Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The PD-L1 inhibitors can be used in combination with one or more other anti-cancer agent(s). In some embodiments, the anti-cancer agent(s) are enzyme/protein/receptor inhibitors or one or more therapies for the treatment of diseases, such as cancer. When a small molecule therapy is described, the term is intended to include any pharmaceutically acceptable salts of such therapies. Examples of diseases and indications treatable with combination therapies include those as described herein. Examples of cancers include solid tumors and liquid tumors, such as blood cancers. For example, the PD-L1 inhibitors can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, PI3K (alpha, beta, gamma, delta), CSFIR, KIT, FLK-II, KDR/FLK-1, FLK-4, flt-1, FGFR1, FGFR2, FGFR3, FGFR4, c-Met, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the PD-L1 inhibitors can be combined with one or more of the following inhibitors for the treatment of cancer. Non-limiting examples of inhibitors that can be combined with the PD-L1 inhibitors for treatment of cancer include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), an IDO inhibitor (e.g., epacadostat, NLG919, or BMS-986205), an LSD1 inhibitor (e.g., INCB59872 and INCB60003), a TDO inhibitor, a PI3K-delta inhibitor (e.g., INCB50797 and INCB50465), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), a poly ADP ribose polymerase (PARP) inhibitor such as rucaparib, olaparib, niraparib, veliparib, or talazoparib, an arginase inhibitor (INCB01158), and an adenosine receptor antagonist or combinations thereof.

The PD-L1 inhibitors can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, Toll receptor agonists, STING agonists, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomy cin C, mitotane, mitoxantrone, nandrolone phenpropionate, nelarabine, nofetumomab, olaparib, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, rucaparib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, niraparib, veliparib, talazoparib and zoledronate.

In some embodiments, the anti-cancer agent is bevacizumab, nivolumab, pembrolizumab, atezolizumab, durvalumab, avelumab, cemiplimab, atezolizumab, avelumab, tislelizumab, spartalizumab, cetrelimab, cetuximab, ramurcirumab, toripalimab, camrelizumab, sintilimab, ipilimumab, retinfanlimab, bleomycin sulfate, topotecan hydrochloride, irinotecan hydrochloride, capecitabine, oxaliplatin, cisplatin, 5-FU (flurouracil), ipilimumab, leucovorin (leucovorin calcium, panitumumab, regorafenib, ziv-afilbercept, paclitaxel, doxetaxel, carboplatin, cisplatin, hydroxyurea, methotrexate sodium, gemcitabine, and combinations thereof. In some embodiments, the anti-cancer agent doxcetaxel, cisplatin, and 5-fluorouracil (5-FU) (TPF); trifluridine and tipracil hydrochloride; carboplatin-paclitaxel; gemcitabine-cisplatin; 5-fluorouracil (5-FU) and mitomycin; 5-fluorouracil (5-FU) and cisplatin; 5-fluorouracil (5-FU), leucovorin calcium (folinic acid), oxaliplatin (FOLFOX); capecitabine and oxaliplatin (CAPOX or XELOX); 5-fluorouracil (5-FU), leucovorin calcium (folinic acid), irinotecan (FOLFIRI); FOLFIRI and bevacizumab; FOLFIRI and cetuximab; 5-fluorouracil (5-FU) and leucovorin calcium (folinic acid) (FU-LV); or capecitabine and irinotecan hydrochloride (XELIRI).

Other anti-cancer agent(s) include antibody therapeutics such as trastuzumab (Herceptin) or antibodies to cytokines (IL-10, TGF-β, etc.).

PD-L1 inhibitors can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A;also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469.In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12.. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, RO7009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The PD-L1 inhibitors can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

The PD-L1 inhibitors can further be used in combination with one or more antiinflammatory agents, steroids, immunosuppressants or therapeutic antibodies.

The PD-L1 inhibitors or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The PD-L1 inhibitors or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi’s Herpes Sarcoma Virus (KHSV). In some embodiments, the PD-L1 inhibitors can be used in combination with tumor specific antigen such as heat shock proteins isolated from tumor tissue itself. In some embodiments, the PD-L1 inhibitors or any of the formulas as described herein, a compound as recited in any of the claims and described herein, or salts thereof can be combined with dendritic cells immunization to activate potent anti-tumor responses.

The PD-L1 inhibitors can be used in combination with bispecific macrocyclic peptides that target Fe alpha or Fe gamma receptor-expressing effectors cells to tumor cells. The PD-L1 inhibitors can also be combined with macrocyclic peptides that activate host immune responsiveness.

The PD-L1 inhibitors can be used in combination with bone marrow transplant for the treatment of a variety of tumors of hematopoietic origin.

The PD-L1 inhibitors can be used in combination with vaccines, to stimulate the immune response to HPV. An example of an HPV vaccine is Gardasil® (Human Papillomavirus Quadrivalent (Types 6, 11, 16, 18)).

When more than one pharmaceutical agent is administered to a patient, they can be administered simultaneously, separately, sequentially, or in combination (e.g., for more than two agents).

### Formulation, Dosage Forms and Administration

When employed as pharmaceuticals, the PD-L1 inhibitors can be administered in the form of pharmaceutical compositions. Thus the present disclosure provides a composition comprising a compound of Formula (I) or any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

The PD-L1 inhibitors also may be provided as pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102®. In some embodiments, the lactose monohydrate is Fast-flo 316®. In some embodiments, the hydroxypropyl methylcellulose is hydroxypropyl methylcellulose 2208 K4M (e.g., Methocel K4 M Premier®) and/or hydroxypropyl methylcellulose 2208 K100LV (e.g., Methocel K00LV®). In some embodiments, the polyethylene oxide is polyethylene oxide WSR 1105 (e.g., Polyox WSR 1105®).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient’s symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 µg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 ug/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### Labeled Compounds and Assay Methods

The PD-L1 inhibitors can further be useful in investigations of biological processes in normal and abnormal tissues. Thus, another aspect of the present disclosure relates to labeled compounds of the disclosure (radio-labeled, fluorescent-labeled, etc.) that would be useful not only in imaging techniques but also in assays, both in vitro and in vivo, for localizing and quantitating PD-1 or PD-L1 protein in tissue samples, including human, and for identifying PD-L1 ligands by inhibition binding of a labeled compound.

The present disclosure further includes isotopically-substituted compounds of the disclosure. An “isotopically-substituted” compound is a compound of the disclosure where one or more atoms are replaced or substituted by an atom having the same atomic number but a different atomic mass or mass number, e.g., a different atomic mass or mass number from the atomic mass or mass number typically found in nature (i.e., naturally occurring). It is to be understood that a “radio-labeled” compound is a compound that has incorporated at least one isotope that is radioactive (e.g., radionuclide). Suitable radionuclides that may be incorporated in PD-L1 inhibitors include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro PD-L1 protein labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

In some embodiments the radionuclide is selected from the group consisting of ³H, ¹⁴C, ¹²⁵I, ³⁵S and ⁸²Br. Synthetic methods for incorporating radio-isotopes into organic compounds are known in the art.

Specifically, a labeled compound of the disclosure can be used in a screening assay to identify and/or evaluate compounds. For example, a newly synthesized or identified compound (i.e., test compound) which is labeled can be evaluated for its ability to bind a PD-L1 protein by monitoring its concentration variation when contacting with the PD-L1 protein, through tracking of the labeling. For example, a test compound (labeled) can be evaluated for its ability to reduce binding of another compound which is known to bind to a PD-L1 protein (i.e., standard compound). Accordingly, the ability of a test compound to compete with the standard compound for binding to the PD-L1 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, e.g., in the treatment or prevention of diseases or disorders associated with the activity of PD-L1 including its interaction with other proteins such as PD-1 and B7-1 (CD80), such as cancer, which include one or more containers containing a pharmaceutical composition comprising a therapeutically effective amount of a compound of Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results. The compounds of the Examples have been found to inhibit the activity of PD-⅟PD-L1 protein/protein interaction according to at least one assay described herein.

### EXAMPLES

### Example 1A. Homogeneous Time-Resolved Fluorescence (HTRF) PD-⅟PD-L1 Binding Assay

The assays were conducted in a standard black 384-well polystyrene plate with a final volume of 20 uL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a His-tag at the C-terminus was purchased from AcroBiosystems (PD1-H5229). Recombinant human PD-1 protein (25-167) with Fc tag at the C-terminus was also purchased from AcroBiosystems (PD1-H5257). PD-L1 and PD-1 proteins were diluted in the assay buffer and 10 uL was added to the plate well. Plates were centrifuged and proteins were preincubated with inhibitors for 40 minutes. The incubation was followed by the addition of 10 uL of HTRF detection buffer supplemented with Europium cryptate-labeled anti-human IgG (PerkinElmer-AD0212) specific for Fc and anti-His antibody conjugated to SureLight®-Allophycocyanin (APC, PerkinElmer-AD0059H). After centrifugation, the plate was incubated at 25° C. for 60 min. before reading on a PHERAstar FS plate reader (665 nm/620 nm ratio). Final concentrations in the assay were - 3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration using the GraphPad Prism 5.0 software.

### Example 1B. Src Homology Region 2 Domain-containing Phosphatase (SHP) Assay

U2OS/PD-L1 cells (DiscoveRx Corporation) were maintained in McCoy’s 5A medium with addition of 10% FBS, 0.25 µg/mL Puromycin. After removing the culture media, the cell medium was replaced with assay medium (RPMI1640 medium with 1% FBS). The U2OS/PD-L1 cells were then added in 384-well black clear bottom assay plate (CELLCOAT® Tissue Culture Plates, Greiner Bio-One) at 5000 cells per well in 20 uL assay medium. Test compounds were prepared by serial dilution in DMSO and 125 nL compound were first transferred to the 384 REMP plate well (Thermofisher) by ECHO liquid handler (Labcyte) followed with addition of 27.5 uL assay medium. 5 µL/well compounds in the assay medium were transferred to the cell plate with 0.05% DMSO in the final assay at 0.25 µM. Jurkat-PD-1-SHP cells (DiscoveRx Corporation) were cultured in RPMI1640 medium supplemented with 10% FBS, 250 µg/mL Hygromycin B, 500 µg/mL G418. After the replacement of culture media with assay medium, 5,000 Jurkat-PD-1-SHP cells in 20 µL were dispensed into each well. The assay plate was incubated at 37° C., 5% CO₂ for 2 hours before 2.5 uL PathHunter reagent 1 (DiscoveRx Corporation) were added to each well. The assay plate was shaken for 1 min at 350 rpm in the dark followed with addition of 10 uL PathHunter reagent 2 (DiscoveRx Corporation). Chemiluminescent signal was recorded with TopCount reader (Perkin Elmer) after incubation at room temperature for 1 hour. Wells with DMSO were served as the positive controls and wells containing no cells were used as negative controls. IC₅₀ determination was performed by fitting the curve of percentage of control activity versus the log of the compound concentration using the GraphPad Prism 6.0 software.

### Example 1C. Nuclear Factor of Activated T-cells (NFAT) Assay

PD-L1 aAPC/CHO-K1cells (Promega) were maintained in F-12 medium with addition of 10% FBS, 200 µg/mL Hygromycin B, 250 µg/mL Geneticin (G418). Jurkat-PD-1-NFAT effector cells (Promega) were cultured in RPMI 1640 medium supplemented with 10% FBS, 100 µg/mL Hygromycin B, 500 µg/mL G418. The culture media of PD-L1 aAPC/CHO-K1 cells were first replaced with assay medium (RPMI1640 medium with 1% FBS). The PD-L1 aAPC/CHO-K1cells were then added in a white 384-well white clear bottom assay plate (CELLCOAT® Tissue Culture Plates, Greiner Bio-One) at 8000 per well in 10 uL assay medium. Test compounds were prepared by serial dilution in DMSO and 0.8 uL test compounds in DMSO were first transferred to the 384 REMP plate well (Thermofisher) by PlateMate Plus (Thermofisher) followed with addition of 50 uL plating medium. 5 uL compounds in the assay medium were transferred to the cells with 0.4% DMSO in the final assay at 2 µM. After removing the culture media, 10,000 Jurkat-PD-1-NFAT effector cells in 5 µL assay medium was dispensed into each well. The assay plate was incubated at 37° C., 5% CO₂ for 24 hours. After the assay plate was equilibrated to room temp for 15 minutes, 20 µL/well of Bio-Glo® reagent (Promega) were added. After 8 minutes incubation at room temperature, luminescence was read at with Pherastar microplate reader (BMG Labtech). The fold of induction (FOI) was calculated based on the ratio of luminescence normalized to the DMSO wells within each assay plate. The maximum percentage of induction was reported based on the ratio between the highest FOI of each compound and the maximum FOI of control compound within each assay plate. Wells with DMSO were served as the negative controls and wells containing control compound with the highest FOI were used as positive controls. EC₅₀ determination was performed by fitting the curve of percent control activity versus the log of the compound concentration using the GraphPad Prism 6.0 software.

### Example 1D. PD-L1 Whole Blood Internalization Assay

To determine PD-L1 internalization in human whole blood, normal human blood (Biological Specialty Corp, Colmar. PA) was incubated in the presence or absence of a concentration range of test compounds and 1 ng/mL human interferon γ (R&D Systems Inc. Minn. MN) in a 96 well “2 mL Assay Block” (Corning, Corning NY) for 18-20 hours at 37⁰C. Blood was then stained with PD-L1 (MIH1, eBioscience; or BD Biosciences San Jose, CA), CD14 (Life Technologies, Carlsbad, CA) for 30 minutes in the dark at room temperature. Whole Blood/red cells were lysed/fixed (lysis buffer BD Biosciences) for 5 minutes at 37⁰C in the dark and then centrifuged at 1600 RPM for 5 minutes. Cells were resuspended in Stain Buffer (BD Bioscience, San Jose, CA) and transferred into 96 well round bottom plates (Coming). Cells were gated on CD14+ (BD Biosciences) and PD-L1 expression determined by mean fluorescence intensity (MFI) (BD LSRFortessa® X-20). IC₅₀ determination was performed by fitting the curve of compound percent inhibition versus the log of the compound concentration using the GraphPad Prism 7.0 software.

### Results

The compounds of Table 1 were assessed in the HTRF PD-⅟PD-L1 binding assay (Example 1A), SHP assay (Example 1B), NFAT assay (Example 1B), and whole blood internalization assay, 24 hour (Example 1D) and results are shown in Table 2 below. + indicates the value was <10 nM; ++ indicates the value was <100 nM; and +++ indicates the value was <500 nM.

TABLE 2

| Compound No. | HTRF binding IC₅₀ (nM) | SHP IC₅₀ (nM) | NFAT EC₅₀ (nM) | Whole Blood IC₅₀ (nM) |
|---|---|---|---|---|
| 1 | + | + | ++ | +++ |
| 2 | + | ++ | +++ | |
| 3 | + | + | ++ | +++ |
| 4 | + | ++ | ++ | +++ |
| 5 | + | + | ++ | +++ |
| 6 | + | + | ++ | ++ |
| 7 | + | + | ++ | + |
| 8 | + | ++ | ++ | |
| 9 | + | + | ++ | |
| 10 | + | + | ++ | ++ |
| 11 | + | + | ++ | ++ |
| 12 | + | + | ++ | ++ |
| 13 | + | + | ++ | ++ |
| 14 | + | ++ | +++ | |
| 15 | + | + | ++ | +++ |
| 16 | + | + | ++ | ++ |
| 17 | + | + | ++ | ++ |
| 18 | + | + | ++ | ++ |
| 19 | + | + | ++ | ++ |
| 20 | + | + | +++ | +++ |
| 21 | + | + | ++ | ++ |
| 22 | + | + | ++ | +++ |
| 23 | + | + | ++ | ++ |
| 24 | + | + | + | ++ |
| 25 | + | + | ++ | ++ |
| 26 | + | + | ++ | ++ |
| 27 | + | + | + | ++ |
| 28 | + | + | + | ++ |
| 29 | + | + | + | ++ |
| 30 | + | + | ++ | ++ |
| 31 | + | + | ++ | ++ |
| 32 | + | + | ++ | ++ |
| 33 | + | + | ++ | ++ |
| 34 | + | + | + | + |

### Example 2. Phase 1 Open-Label Clinical Trial

This study is a Phase 1, open-label, dose-finding study for Compound 1 (see Table 1, supra). Eligible participants enrolled will receive Compound 1 as an oral tablet (50-mg, 100-mg, or 400-mg) during 21-day or 28-day cycles until disease progression, unacceptable toxicity, death, withdrawal of consent, or any other reason. Continuous treatment administration may be once daily (QD) or twice daily (BID).

Following a dose escalation study to identify a pharmacological active dose, this trial will enroll up to 60 participants with HPV-positive tumors at 1 or more pharmacologically active dose (PAD) doses to further evaluate safety, efficacy, PK, and pharmacodynamic effects. A maximum of 20 participants may be enrolled per dose level. Participants must have HPV-positive tumors as determined by a local laboratory using p16 IHC, polymerase chain reaction methods, or other locally-available method to detect HPV.

Participants are eligible to be included in the study only if all of the following criteria apply:
1. Male and female participants must be ≥ 18 years of age at the time of signing the ICF.
2. Ability to comprehend and willingness to sign an ICF.
3. Must be willing and able to conform to and comply with all Protocol requirements, including all scheduled visits, Protocol procedures, and the ability to swallow oral medication.
4. Histologically confirmed advanced solid tumors with measurable lesions (per RECIST v1.1 or RANO for primary brain tumors) that are considered nonamenable to surgery or other curative treatments or procedures. Tumor lesions located in a previously irradiated area or in an area subjected to other locoregional therapy are considered measureable if progression has been demonstrated in the lesion.
5. Participants must have disease progression after treatment with available therapies that are known to confer clinical benefit or who are intolerant to or ineligible for standard treatment.
6. Willingness to undergo a tumor biopsy to obtain tumor tissue.
7. Eastern Cooperative Oncology Group performance status score of 0 or 1.
8. Life expectancy > 12 weeks.
9. Willingness to avoid pregnancy or fathering children.
10. For the cohort participating in the extension to participants with HPV-positive solid tumors: any HPV-positive solid tumor which has received prior standard therapy.

### Results

Adult patients (≥18 years) with advanced solid tumors were enrolled into this open-label study. Patients had disease progression after standard available therapy, or were intolerant of, or ineligible for standard treatment. Measureable disease was required. A modified 3+3 dose escalation design was employed, followed by dose expansions. The primary endpoints were safety and tolerability of Compound 1, identification of a pharmacologically active dose and/or maximum tolerated dose, and confirmation of the recommended Phase 2 dose. Secondary endpoints included PK, pharmacodynamics, and efficacy as assessed by investigator-determined objective response and disease control rates (complete response (CR), partial response (PR), or stable disease (SD)).

Table 3 shows the response rate for fourteen (14) patients that were evaluated for response, receiving 400 mg BID or 800 mg BID of Compound 1 (patients received 400 mg BID of Compound 1 unless otherwise indicated). Patients that were confirmed to have HPV+ or confirmed to be HPV- are indicated below. Patients of unknown HPV status are also indicated. The CDC has estimated that certain cancers are more probable to be HPV+, including anal cancer (91%), cervical cancer (91%), penile cancer (63%), and vaginal cancer (75%) (percentages indicate the percentage of cancers that are probably caused by any HPV type; see https://www.cdc.gov/cancer/hpv/statistics/cases.htm). Hence, the ORR based on PR or CR is estimated to be about 28.6%, factoring in the responses in patient with unknown HPV status.

TABLE 3

Compound 1 Treatment

| | | Anal | HNSCC | Cervical | Penile | Vaginal | Total |
|---|---|---|---|---|---|---|---|
| # patients evaluable for response | | 8 | 1 | 3 | 1 | 1 | 14 |
| PR/CR | Total | 3 | | | | 1 | 4 |
| | Confirmed HPV+ | 1^{a} | | | | 1 | 2 |
| | Unknown | 2^{b} | | | | | 2 |
| | Confirmed HPV- | | | | | | |
| SD | Total | 2 | | 1 | 1 | | 4 |
| | Confirmed HPV+ | 1 | | 1 | 1 | | 3 |
| | Unknown | 1 | | | | | 1 |
| | Confirmed HPV- | | | | | | |
| PD | Total | 3 | 1 | 2 | | | 6 |
| | Confirmed HPV+ | 1 | | 2 | | | 3 |
| | Unknown | 2 | | | | | 2 |
| | Confirmed HPV- | | 1^{a} | | | | 1 |

^{a}Patient received 800 mg BID of Compound 1
b¹ out of 2 patients received 800 mg BID of Compound 1.

### Example 3. Phase 1 Open-Label Clinical Trials for Compounds 24 and 30

Two open-label, nonrandomized, global multicenter Phase 1 studies in participants with select advanced solid tumors are being conducted for Compounds 24 and 30 in Table 1 supra. In addition to the primary endpoint of determining safety, tolerability, PAD and/or maximum tolerated dose (MTD) of each compound, the studies are exploring preliminary efficacy (complete response (CR), partial response (PR), stable disease (SD), or progressive disease (PD)) in participants with select advanced cancers, including virally mediated tumors such as HPV+ carcinomas. HPV+ tumors can be determined by a local laboratory using p16 IHC, polymerase chain reaction methods, or other locally-available method to detect HPV. Results for patients having solid tumors whose HPV+ status was confirmed, suspected or negative are shown below for Compound 24 and Compound 30 in Tables 4 and 5, respectively. Dose levels used for each of Compound 24 and Compound 30 are shown in the tables.

TABLE 4

Compound 24 Treatment

| Patient | Dose Level | Tumor Type | HPV/p16 status | Best Overall Response |
|---|---|---|---|---|
| 1 | 200 mg QD | Cervical cancer (AC) | Not determined | SD |
| 2 | 200 mg BID | Cervical cancer (clear cell) | Not determined | PD |
| 3 | 400 mg BID | Anal carcinoma | HPV 16 positive | SD |
| 4 | 400 mg BID | Anal carcinoma | HPV 16 positive | SD |
| 5 | 300 mg BID | Vaginal cancer (SCC) | HPV 16 positive | SD |
| 6 | 400 mg BID | Cervical cancer (SCC) | HPV 16 positive | PR |
| 7 | 200 mg BID | Anal carcinoma (SCC) | Positive unknown type | PD |
| 8 | 400 mg BID | Anal carcinoma | HPV 16 positive | PD |
| 9 | 600 mg QD | Anal carcinoma (rectovaginal) | HPV 16,33,31 positive | SD |
| 10 | 400 mg BID | Cervical cancer (AC) | HPV 16 positive | SD |
| 11 | 400 mg BID | Anal carcinoma (SCC) | Not determined | PR |
| 12 | 600 mg QD | Anal carcinoma (SCC) | Not determined | SD |
| 13 | 400 mg BID | Rectovaginal septum (SCC) | P16+, HPV 16 | PD |
| 14 | 600 mg QD | Anal carcinoma | HPV 16 | PD |
| 15 | 800 mg QD | Anal carcinoma | HPV 16 | PD |
| 16 | 800 mg QD | Vulvar (SCC) | Not determined | PD |
| 17 | 400 mg BID | Anal cancer | Negative | Not assessed |
| 18 | 800 mg QD | Cervical cancer (SCC) | P16 positive | PD |
| 19 | 800 mg QD | Anal cancer | HPV 16 positive | PD |
| 20 | 800 mg QD | Anal cancer | HPV 16 positive | PD |
| 21 | 300 mg BID | Anal cancer | HPV 16 | SD |
| 22 | 300 mg BID | Cervical cancer | Not determined | Not yet due for scan |
| 23 | 400 mg BID | Cervical cancer | Not determined | Not yet due for scan |

TABLE 5

Compound 30 Treatment

| Patient | Dose Level | Tumor Type | HPV/p16 status | Best Overall Response |
|---|---|---|---|---|
| 1 | 300 mg BID | Cervical cancer (SCC) | p16 positive | PR |
| 2 | 300 mg BID | Nasopharyngeal (SCC) | p16 negative | SD |
| 3 | 300 mg BID | Cervical cancer (SCC) | p16 positive | SD |
| 4 | 400 mg BID | Cervical cancer (SCC) | p16 positive | PD |
| 5 | 400 mg BID | Nasopharyngeal (SCC) | Not determined | SD |
| 6 | 400 mg BID | Cervical cancer (SCC) | Positive due to HSIL | SD |
| 7 | 400 mg BID | Acinic cell parotid gland carcinoma | Not determined | PD |
| 8 | 400 mg BID | Cervical cancer (adenosquamous) | p16 positive | PR |
| 9 | 600 mg QD | Cervical cancer (SCC) | p16 positive | PD |
| 10 | 600 mg QD | Cervical cancer (SCC and adenocarcinoma) | Not determined | PD |
| 11 | 600 mg BID | Cervical cancer (SCC) | p16 positive | PD |
| 12 | 600 mg BID | Penile cancer (SCC) | p16 positive | SD |
| 13 | 200 mg BID | Anal canal (SCC) | Not determined | PD |
| 14 | 600 mg BID | Cervical cancer (adenocarcinoma) | HPV 18 positive | Not yet due for scan |
| 15 | 600 mg BID | Cervical cancer (SCC) | Not determined | Not yet due for scan |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A method of treating a human papillomavirus (HPV)-positive cancer in a human subject in need thereof, comprising administering to the human subject a small molecule PD-L1 inhibitor.

2. The method of claim 1, wherein the cancer has been previously determined to be HPV-positive.

3. The method of claim 1, or wherein the HPV-positive cancer is positive for at least one of HPV type 16, 18, 31, 33, 35, 39, 45, 51, 52, 56, 58, 59, 66, or 68.

4. The method of claim 1, or wherein the HPV-positive cancer is positive for HPV type 16 and/or 18.

5. The method of claim 1, wherein the HPV-positive cancer is positive for p16.

6. A method of treating an HPV-positive cancer in a human subject in need thereof, comprising:
identifying a sample obtained from the human subject as positive for HPV; and
administering to the human subject a small molecule PD-L1 inhibitor.

7. The method of claim 6, comprising identifying the sample as positive for at least one of HPV type 16, 18, 31, 33, 35, 39, 45, 51, 52, 56, 58, 59, 66, or 68.

8. The method of claim 6, comprising identifying the sample as positive for HPV type 16 and/or 18.

9. The method of claim 6, comprising identifying the sample as positive for HPV type 16.

10. The method of claim 6, comprising identifying the sample as positive for HPV type 18.

11. The method of claim 6, wherein the method comprises detecting an HPV nucleic acid in the sample.

12. The method of claim 6, wherein the method comprises detecting an HPV protein in the sample.

13. The method of claim 12, wherein the method comprises identifying the sample as positive for HPV by p16 immunohistochemistry.

14. The method of claim 1, wherein the HPV-positive cancer is a solid tumor.

15. The method of claim 14, wherein the solid tumor is selected from the group consisting of anal cancer, cervical cancer, vaginal cancer, a head and neck cancer, vulvar cancer, rectal cancer, penile cancer, rectovaginal cancer, nasopharyngeal cancer, and acinic cell parotid gland carcinoma.

16-24. (canceled)

25. The method of claim 1, wherein the PD-L1 inhibitor has a molecular weight of less than 1000 daltons.

26. The method of claim 1, wherein the PD-L1 inhibitor is a compound of Formula (I): 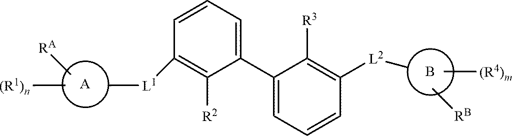
or a pharmaceutically acceptable salt thereof, wherein:
ring A is selected from 5-10-membered heteroaryl, having carbon ring members and 1, 2, 3, or 4 heteroatom ring members selected from N, O and S; and 4-10-membered heterocycloalkyl, having carbon ring members and 1, 2, 3, or 4 heteroatom ring members selected from N, O and S;
ring B is selected from 5-10-membered heteroaryl, having carbon ring members and 1, 2, 3, or 4 heteroatom ring members selected from N, O and S; and 4-10-membered heterocycloalkyl, having carbon ring members and 1, 2, 3, or 4 heteroatom ring members selected from N, O and S;
L¹ is a bond, —C(O)NH—, —NHC(O)-, and —NH—;
L² is a bond, —C(O)NH—, —NHC(O)-, and —NH-;
each R¹ is independently halo, CN, C₁₋₃ alkyl, C₁₋₃ alkoxy, C₁₋₃ haloalkyl, C₁₋₃ haloalkoxy, amino, C₁₋₃ alkylamino, or di(C₁₋₃ alkyl)amino;
R² is halo, CH₃, or CN;
R³ is halo, CH₃, or CN;
each R⁴ is independently halo, CN, C₁₋₃ alkyl, C₁₋₃ alkoxy, C₁₋₃ haloalkyl, C₁₋₃ haloalkoxy, amino, C₁₋₃ alkylamino, or di(C₁₋₃ alkyl)amino;
R^{A} and R^{B} are each independently selected from C₁₋₃ alkyl, C₄₋₇ cycloalkylene-(R^{c}), -(C₁₋₃ alkylene)-R^{c} and -C(=O)-(C₁₋₃ alkylene)-R^{c}, wherein said C₄₋₇ cycloalkylene is optionally substituted by one C₁₋₃ alkyl substituent;
each R^{c}is independently selected from phenyl, C₃₋₇ cycloalkyl, 5-6 membered heteroaryl, 4-10 membered heterocycloalkyl, OR^{a1}, SR^{a1}, NHOR^{a1}, C(O)R^{b1}, C(O)NR^{c1}R^{d1}, C(O)NR^{c1}(OR^{a1}), C(O)OR^{a1}, OC(O)R^{b1}, OC(O)NR^{c1}R^{d1}, NR^{c1}R^{d1}, NR^{c1}NR^{c1}R^{d1}, NR^{c1}C(O)R^{b1}, NR^{c1}C(O)OR^{a1}, NR^{c1}C(O)NR^{c1}R^{d1}, NR^{c1}S(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};wherein the phenyl, C₃₋₇ cycloalkyl, 5-6 membered heteroaryl, and 4-10 membered heterocycloalkyl of R^{C} are each optionally substituted with 1, 2, or 3 independently selected R^{D} substituents;
each R^{a1}, R^{c1}, and R^{d1} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl-, wherein said C₁₋₆ alkyl, C₂₋₆ alkenyl, C₂₋₆ alkynyl, C₁₋₆ haloalkyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl- are each optionally substituted with 1, 2, or 3 independently selected R^{D} substituents;
or, any R^{c1} and R^{d1} attached to the same N atom, together with the N atom to which they are attached, form a 4-10 membered heterocycloalkyl group, wherein the 4-10 membered heterocycloalkyl group is optionally substituted with 1, 2, or 3 independently selected R^{D} sub stituents;
each R^{b1} is independently selected from C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl-, which are each optionally substituted with 1, 2, or 3 independently selected R^{D} substituents;
each R^{D} substituent is independently selected from halo, C₁₋₆ alkyl, C₂₋₆ alkenyl, C₂₋₆ alkynyl, C₁₋₆ haloalkyl, C₁₋₆ haloalkoxy, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, (4-10 membered heterocycloalkyl)-C₁₋₄ alkyl-, CN, OH, NH₂, NO₂, OR^{a2}, SR^{a2}, NHOR^{a2}, C(O)R^{b2}, C(O)NR^{c2}R^{d2}, C(O)NR^{c2}(OR^{a2}), C(O)OR ^{a2}, OC(O)R^{b2}, OC(O)NR^{c2}R^{d2}, NR^{c2}R^{d2}, NR^{c2}NR^{c2}R^{d2}, NR^{c2}C(O)R^{b2}, NR^{c2}C(O)OR^{a2}, NR^{c2}C(O)NR^{c2}R^{d2}, NR^{c2}S(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein the C₁₋₆ alkyl, C₂₋₆ alkenyl, C₂₋₆ alkynyl, C₁₋₆ haloalkyl, C₁₋₆ haloalkoxy, phenyl, C₃₋₇ cycloalkyl, 5-6 membered heteroaryl, 4-10 membered heterocycloalkyl, phenyl-C₁₋₄ alkyl-, C₃₋₇ cycloalkyl-C₁₋₄ alkyl-, (5-6 membered heteroaryl)-C₁₋₄ alkyl-, and (4-10 membered heterocycloalkyl)-C₁₋₄ alkyl- of R^{D} are each further optionally substituted with 1, 2, or 3 independently selected R^{E} substituents;
each R^{a2}, R^{c2}, and R^{d2} is independently selected from H, C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, wherein said C₁₋₆ alkyl, C₂₋₆ alkenyl, C₂₋₆ alkynyl, C₁₋₆ haloalkyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl are each optionally substituted with 1, 2, or 3 independently selected R^{E} substituents;
or, any R^{c2} and R^{d2} attached to the same N atom, together with the N atom to which they are attached, form a 4-7 membered heterocycloalkyl group, wherein the 4-7 membered heterocycloalkyl group is optionally substituted with 1, 2, or 3 independently selected R^{E} substituents;
each R^{b2} is independently selected from C₁₋₆ alkyl, C₁₋₆ haloalkyl, C₂₋₆ alkenyl, C₂₋₆ alkynyl, C₃₋₇ cycloalkyl, phenyl, 4-7 membered heterocycloalkyl, 5-6 membered heteroaryl, C₃₋₇ cycloalkyl-C₁₋₄ alkyl, phenyl-C₁₋₄ alkyl, 4-7 membered heterocycloalkyl-C₁₋₄ alkyl, and 5-6 membered heteroaryl-C₁₋₄ alkyl, which are each optionally substituted with 1, 2, or 3 independently selected R^{E} substituents;
each R^{E} is independently selected from OH, NO₂, CN, halo, C₁₋₃ alkyl, C₂₋₃ alkenyl, C₂₋₃ alkynyl, C₁₋₃ haloalkyl, cyano-C₁₋₃ alkyl, HO-C₁₋₃ alkyl, C₁₋₃ alkoxy-C₁₋₃ alkyl, C₃₋₇ cycloalkyl, C₁₋₃ alkoxy, C₁₋₃ haloalkoxy, amino, C₁₋₃ alkylamino, di(C₁₋₃ alkyl)amino, thio, C₁₋₃ alkylthio, C₁₋₃ alkylsulfinyl, C₁₋₃ alkylsulfonyl, carbamyl, C₁₋₃ alkylcarbamyl, di(C₁₋₃ alkyl)carbamyl, carboxy, C₁₋₃ alkylcarbonyl, C₁₋₃ alkoxycarbonyl, C₁₋₃ alkylcarbonyloxy, C₁₋₃ alkylcarbonylamino, C₁₋₃ alkoxycarbonylamino, C₁₋₃ alkylaminocarbonyloxy, C₁₋₃ alkylsulfonylamino, aminosulfonyl, C₁₋₃ alkylaminosulfonyl, di(C₁₋₃ alkyl)aminosulfonyl, aminosulfonylamino, C₁₋₃ alkylaminosulfonylamino, di(C₁₋₃ alkyl)aminosulfonylamino, aminocarbonylamino, C₁₋₃ alkylaminocarbonylamino, and di(C₁₋₃ alkyl)aminocarbonylamino;
the subscript n is an integer of 0, 1, or 2; and
the subscript m is an integer of 0, 1, or 2.

27. The method of claim 26, wherein Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl; and Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, 5,6-dihydro-4H-pyrrolo[3,4-d]thiazol-2-yl, and pyrazin-2-yl.

28. The method of claim 26, wherein Ring A is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl; and Ring B is selected from benzo[d]oxazol-5-yl, naphthyridin-8-yl, pyrido[3,2-d]pyrimidin-4-yl, [1,2,4]triazolo[1,5-a]pyridin-2-yl, pyridin-2-yl, 4,5,6,7-tetrahydro-1H-imidazo[4,5-c]pyridin-2-yl, and 5,6-dihydro-4H-pyrrolo[3,4-d]thiazol-2-yl.

29. The method of claim 26, wherein each R¹ and R⁴ are independently CHF₂, CN, Cl, OCH₃, or CH₃.

30. The method of claim 26, wherein each R¹ and R⁴ are independently CHF₂, CN, Cl, or CH₃.

31. The method of claim 26, wherein R² and R³ are independently Cl, CH₃, or CN.

32. The method of claim 26, wherein n is 0 or 1; and m is 0 or 1.

33. The method of claim 26, wherein R^{A} and R^{B} are each independently selected from CH₃, -CH₂-R^{c}, -CH₂CH₂-R^{c}, -CH₂-CH(R^{c})-CH₃, -(cyclohex-1,4-diyl)-(R^{C}), and -C(=O)-CH₂-R^{C}, wherein said cyclohex-1,4-diyl is optionally substituted by one methyl substituent.

34. The method of claim 26, wherein each R^{C} is independently selected from OR^{a1}, NR^{c1}R^{d1}, cyclohexyl, bicyclo[2.2.1]heptanyl, pyrrolidinyl, and piperidinyl, wherein the cyclohexyl, bicyclo[2.2.1]heptanyl, pyrrolidinyl, and piperidinyl of R^{C} are each optionally substituted with 1 or 2 independently selected R^{D} substituents.

35. The method of claim 26, wherein each R^{a1} is H; and each R^{c1} and R^{d1} is independently selected from H, methyl, ethyl, and 2-oxo-pyrrolidinylmethyl, wherein said methyl and ethyl are each optionally substituted with 1 or 2 independently selected R^{D} substituents.

36. The method of claim 26, wherein each R^{a1} is H; and each R^{c1} and R^{d1} is independently selected from H, methyl, and ethyl, wherein said methyl and ethyl are each optionally substituted with 1 or 2 independently selected R^{D} substituents.

37. The method of claim 26, wherein each R^{D} is independently selected from OH, CO₂H, and CH₃.

38. The method of claim 26, wherein each R^{D} is independently selected from OH and CO₂H.

39. The method of claim 26, wherein the PD-L1 inhibitor is selected from:
(R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
N-(2-chloro-3′-(8-chloro-6-((2-hydroxyethylamino)methyl)-[1,2,4]triazolo[1,5-a]pyridin-2-yl)-2′-methylbiphenyl-3-yl)-5-((2-hydroxyethylamino)methyl)picolinamide;
(S)-1 -((7-cyano-2-(3 ‘-(3 -(((S)-3 -hydroxypyrrolidin-1 -yl)methyl)- 1,7-naphthyridin-8-ylamino)-2,2′-dimethylbiphenyl-3-yl)benzo[d]oxazol-5-yl)methyl)pyrrolidine-3-carboxylic acid;
(R)-1-((7-cyano-2-(3′-(3-(((S)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
(S)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid;
1-((7-cyano-2-(3′-(5-(2-(dimethylamino)acetyl)-5,6-dihydro-4H-pyrrolo[3,4-d]thiazol-2-yl)-2,2′-dimethylbiphenyl-3-yl)benzo[d]oxazol-5-yl)methyl)piperidine-4-carboxylic acid
N,N′-(2-chloro-2′-methylbiphenyl-3,3′-diyl)bis(5-((2-hydroxyethylamino) methyl)picolinamide);
(R)-1-((6-(2′-chloro-3′-(5-((3-hydroxypyrrolidin-1-yl)methyl)picolinamido)-2-methylbiphenyl-3-ylcarbamoyl)pyridin-3-yl)methyl)piperidine-4-carboxylic acid
(S)-1-((6-((2′-chloro-2-methyl-3′-(5-(pyrrolidin-1-ylmethyl)picolinamido)-[1,1′-biphenyl]-3-yl)carbamoyl)-4-methylpyridin-3-yl)methyl)piperidine-2-carboxylic acid;
trans 4-(2-(2-(2-chloro-3′-(3-(((R)-3-hydroxypyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)ethyl)cyclohexanecarboxylic acid;
cis-4-((2-(2-chloro-3′-(3-(((R)-3-hydroxy-3-methylpyrrolidin-1-yl)methyl)-1,7-naphthyridin-8-ylamino)-2′-methylbiphenyl-3-ylcarbamoyl)-1-methyl-6,7-dihydro-1H-imidazo[4,5-c]pyridin-5(4H)-yl)methyl)cyclohexanecarboxylic acid;
(R)-4-(2-(2-chloro-3′-(7-((3-hydroxypyrrolidin-1-yl)methyl)pyrido[3,2-d]pyrimidin-4-ylamino)-2′-methylbiphenyl-3-ylcarbamoyl)-1-methyl-6,7-dihydro-1H-imidazo[4,5-c]pyridin-5(4H)-yl)-1-methylcyclohexanecarboxylic acid;
(R)-1-((8-((2-chloro-3′-(5-(N-ethyl-N-methylglycyl)-5,6-dihydro-4H-pyrrolo[3,4-d]thiazol-2-yl)-2′-methyl-[1,1′-biphenyl]-3-yl)amino)-1,7-naphthyridin-3-yl)methyl)pyrrolidine-3-carboxylic acid;
(R)-2-(dimethylamino)-1-(2-(3′-(5-(2-(3-hydroxypyrrolidin-1-yl)acetyl)-5,6-dihydro-4H-pyrrolo[3,4-d]thiazol-2-yl)-2,2′-dimethylbiphenyl-3-yl)-4H-pyrrolo[3,4-d]thiazol-5(6H)-yl)ethanone;
trans-4-((2-(2′-chloro-3′-(1,5-dimethyl-4,5,6,7-tetrahydro-1H-imidazo[4,5-c]pyridine-2-carboxamido)-2-methylbiphenyl-3 -ylcarbamoyl)-1-methyl-6,7-dihydro-1H-imidazo[4, 5-c]pyridin-5(4H)-yl)methyl)cyclohexane-1-carboxylic acid;
trans-4-(2-(2-((2′-chloro-3′-(1,5-dimethyl-4,5,6,7-tetrahydro-1H-imidazo[4,5-c]pyridine-2-carboxamido)-2-methyl-[1,1‘-biphenyl]-3-yl)carbamoyl)-1-methyl-1,4,6,7-tetrahydro-5H-imidazo[4,5-c]pyridin-5-yl)ethyl)cyclohexane-1-carboxylic acid;
cis-4-((2-((2′-chloro-3′-(1,5-dimethyl-4,5,6,7-tetrahydro-1H-imidazo[4,5-c]pyridine-2-carboxamido)-2-methyl-[1,1′-biphenyl]-3-yl)carbamoyl)-1-methyl-1,4,6,7-tetrahydro-SH-imidazo[4,5-c]pyridin-5-yl)methyl)cyclohexane-1-carboxylic acid;
cis-4-((2-((2-chloro-3′-(1,5-dimethyl-4,5,6,7-tetrahydro-1H-imidazo[4,5-c]pyiidine-2-carboxamido)-2′-methyl-[1,1′-biphenyl]-3-yl)carbamoyl)-1-methyl-1,4,6,7-tetrahydro-SH-imidazo[4,5-c]pyridin-5-yl)methyl)cyclohexane-1-carboxylic acid;
trans-4-(2-(2-((2′-chloro-2-cyano-3′-(1,5-dimethyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-SH-imidazo[4,5-c]pyridin-5-yl)ethyl)cyclohexane-1-carboxylic acid;
trans-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid;
cis-4-((2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)methyl)cyclohexane-1-carboxylic acid;
4-(2-(2-(2-chloro-3′-(5-(2-(ethyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid;
4-(2-(2-(2-chloro-3′-(5-(2-(isopropyl(methyl)amino)acetyl)-5,6-dihydro-4H-pyrrolo[3,4-d]thiazol-2-yl)-2′-methylbiphenyl-3-ylcarbamoyl)-1-methyl-6,7-dihydro-1H-imidazo[4,5-c]pyridin-5(4H)-yl)ethyl)cyclohexane-1-carboxylic acid;
(R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)pyrrolidine-3-carboxylic acid;
(R)-1-((7-cyano-2-(3′-(2-(difluoromethyl)-7-(((R)-3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)-3-methylpyrrolidine-3-carboxylic acid;
(R)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(S)-1-((7-cyano-2-(3′-(2-(difluoromethyl)-7-((3-hydroxy-3-methylpyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid;
(R)-4-(2-(2-((2,2′-dichloro-3′-(5-(2-hydroxypropyl)-1-methyl-4,5,6,7-tetrahydro-1H-imidazo[4,5-c]pyridine-2-carboxamido)-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2. 1]heptane-1-carboxylic acid);
4-((2-((3′-(5-(2-(4-carboxybicyclo[2.2.1]heptan-1-yl)ethyl)-1-methyl-4,5,6,7-tetrahydro-1H-imidazo[4,5-c]pyridine-2-carboxamido)-2,2′-dichloro-[1,1′-biphenyl]-3-yl)carbamoyl)-1-methyl-1,4,6,7-tetrahydro-5H-imidazo[4,5-c]pyridin-5-yl)methyl)bicyclo[2.2.1]heptane-1-carboxylic acid;
4,4′-(((((2-chloro-2′-methyl-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2. 1]heptane-1-carboxylic acid);
4,4′-(((((2-chloro-2′-cyano-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid); and
(R)-4-(2-(2-((2-chloro-3′-((2-(difluoromethyl)-7-((3-hydroxypyrrolidin-1-yl)methyl)pyrido[3,2-d]pyrimidin-4-yl)amino)-2′-methyl-[1,1′-biphenyl]-3-yl)carbamoyl)-1-methyl-1,4,6,7-tetrahydro-5H-imidazo[4,5-c]pyridin-5-yl)ethyl)bicyclo[2.2.1]heptane-1-carboxylic acid;
or a pharmaceutically acceptable salt thereof.

40. The method of claim 26, wherein the PD-L1 inhibitor is (R)-1-((7-cyano-2-(3′-(3-(((R)-3-hydroxypyrrolidin-1-yl)methyl)-1,7-naphthyridin-8-ylamino)-2,2′-dimethylbiphenyl-3-yl)benzo[d]oxazol-5-yl)methyl)pyrrolidine-3-carboxylic acid, or a pharmaceutically acceptable salt thereof.

41. The method of claim 26, wherein the PD-L1 inhibitor is Compound 24 ((R)-1-((7-cyano-2-(3′-(2-(difluoromethyl)-7-((3-hydroxypyrrolidin-1-yl)methyl)pyrido[3,2-d]pyrimidin-4-ylamino)-2,2′-dimethylbiphenyl-3-yl)benzo[d]oxazol-5-yl)methyl)piperidine-4-carboxylic acid), or a pharmaceutically acceptable salt thereof.

42. The method of claim 41, wherein the administering comprises administering 200 mg to 800 mg of Compound 24 twice daily (BID) to the human subject.

43. The method of claim 41, wherein the administering comprises administering 200 mg to 800 mg of Compound 24 once daily (QD) to the human subject.

44. The method of claim 41, wherein the administering comprises administering 200 mg, 300 mg, 400 mg, 500 mg, 600 mg, 700 mg, or 800 mg of Compound 24 twice daily (BID) to the human subject.

45. The method of claim 41, wherein the administering comprises administering 200 mg, 300 mg, 400 mg, 500 mg, 600 mg, 700 mg, or 800 mg of Compound 24 once daily (QD) to the human subject.

46. The method of claim 26, wherein the PD-L1 inhibitor is Compound 30 (4,4′-(((((2,2′-dichloro-[1,1′-biphenyl]-3,3′-diyl)bis(azanediyl))bis(carbonyl))bis(1-methyl-1,4,6,7-tetrahydro-5H-imidazo[4,5-c]pyridine-2,5-diyl))bis(ethane-2,1-diyl))bis(bicyclo[2.2.1]heptane-1-carboxylic acid)), or a pharmaceutically acceptable salt thereof.

47. The method of claim 46, wherein the administering comprises administering 200 mg to 800 mg of Compound 30 twice daily (BID) to the human subject.

48. The method of claim 46, wherein the administering comprises administering 200 mg to 800 mg of Compound 30 once daily (QD) to the human subject.

49. The method of claim 46, wherein the administering comprises administering 200 mg, 300 mg, 400 mg, 500 mg, 600 mg, 700 mg, or 800 mg of Compound 30 twice daily (BID) to the human subject.

50. The method of claim 46, wherein the administering comprises administering 200 mg, 300 mg, 400 mg, 500 mg, 600 mg, 700 mg, or 800 mg of Compound 30 once daily (QD) to the human subject.

51. The method of claim 1, wherein the method further comprises administering to the human subject one or more anti-cancer agents.

52-53. (canceled)
